(19) United States
(12) Patent Application Publication
(10) Pub. No.: US 2026/0185124 A1
(43) Pub. Date: Jul. 2, 2026

(54) NISIN FOR ETHANOL MICROBIAL CONTROL AND CLEANING

(71) Applicant: ECOLAB USA INC., St. Paul, MN (US)

(72) Inventors: Matthew Albert Bilski, New Brighton, MN (US); Brian Lawrence Sholes, North Saint Paul, MN (US); Christopher M. Nagel, Saint Paul, MN (US); Richard Joseph Walsh, Woodbury, MN (US); Junzhong Li, Eagan, MN (US); Joshua Orland Luedtke, Hudson, WI (US); Marina Anne Tschida, Saint Paul, MN (US)

(21) Appl. No.: 19/427,168

(22) Filed: Dec. 19, 2025

Related U.S. Application Data
(60) Provisional application No. 63/739,829, filed on Dec. 30, 2024.
(60) Provisional application No. 63/814,845, filed on May 30, 2025.

(51) Int. Cl. C12P 7/10; A01N 63/50; A01P 1/00

(52) U.S. Cl. CPC C12P 7/10; A01N 63/50; A01P 1/00

(57) ABSTRACT

Compositions, methods, and systems for controlling microbial populations within an ethanol production system. Nisin and other components may be applied to various parts of an ethanol production system, such as of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

### CROSS REFERENCE TO RELATED APPLICATIONS

This application claims the benefit of and priority to U.S. Provisional Application No. 63/739,829 filed Dec. 30, 2024; and U.S. Provisional Application No. 63/814,845 filed May 30, 2025; the disclosures of which are hereby incorporated by reference in their entirety.

### BACKGROUND

In ethanol fermentation, microbial contamination poses significant challenges as unwanted microbial populations may compete with yeast for nutrients, reduce ethanol yields, produce byproducts that inhibit fermentation, and disrupt further processing steps of distillation and evaporation. Ethanol yield and quality may also suffer due to lack of proper cleaning to control microbial contamination. Microbial populations, organic residues, and biofilms can accumulate in fermenters, pipelines, and other equipment, which are difficult to remove. It is against this background that the present disclosure is made.

### SUMMARY

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the embodiments disclosed herein are based.

Described herein is a method of reducing microbial population in an ethanol production system, the method comprising: adding a composition to the ethanol production system, the composition comprising nisin, wherein the composition is added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

Also described herein is a method for controlling microbial populations in an ethanol production system, the method comprising: adding nisin to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, wherein the nisin is the only antimicrobial agent added to the ethanol production system during a fermentation process.

Also described herein is a method for controlling microbial populations in an ethanol production system, the method comprising: adding a composition comprising nisin and at least one surfactant to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

Also described herein is a method of performing CIP cleaning or sanitizing of an ethanol production system, the method comprising: generating a composition; applying the composition to the ethanol production system, the composition comprising nisin; and circulating the composition in the ethanol production system using a CIP system for a period of time.

Also described herein is a system for monitoring microbial population in an ethanol production system, the system comprising: taking a sample from the ethanol production system; measuring the microbial population in the sample; and comparing the measured microbial population to a threshold value.

Also described herein is a method of increasing a rate of ethanol production in an ethanol production system, the method comprising: adding a composition to the ethanol production system, the composition comprising nisin, wherein the composition is added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and wherein the rate of ethanol production in the ethanol production system is increased relative to a system under the same or similar conditions where the composition has not been added.

Also described herein is a method of decreasing an ethanol production time in an ethanol production system, the method comprising: adding a composition to the ethanol production system, the composition comprising nisin, wherein the composition is added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and wherein the ethanol production time in the ethanol production system is decreased relative to a system under the same or similar conditions where the composition has not been added.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1A depicts a schematic diagram of a fermentation system.

FIG. 1B depicts a structural representation of nisin.

FIG. 2 is a graphical representation of the results of Example 1.

FIG. 3 is a graphical representation of the results of Example 1.

FIG. 4 is a graphical representation of the results of Example 1.

FIG. 5 is a graphical representation of the results of Example 1.

FIG. 6 is a graphical representation of the results of Example 3.

FIG. 7 is a graphical representation of the results of Example 3.

FIG. 8 is a graphical representation of the results of Example 3.

FIG. 9 is a graphical representation of the results of Example 4.

FIG. 10 is a graphical representation of the results of Example 4.

FIG. 11 is a graphical representation of the results of Example 4.

FIG. 12 is a graphical representation of the results of Example 5.

FIG. 13 is a graphical representation of the results of Example 5.

FIG. 14 is a graphical representation of the results of Example 5.

FIG. 15 is a graphical representation of the results of Example 6.

FIG. 16 is a graphical representation of the results of Example 6.

FIG. 17 is a graphical representation of the results of Example 6.

FIG. 18 is a graphical representation of the results of Example 6.

FIG. 19 is a graphical representation of the results of Example 7.

FIG. 20 is a graphical representation of the results of Example 7.

FIG. 21 is a graphical representation of the results of Example 7.

FIG. 22 is a graphical representation of the results of Example 7.

FIG. 23 is a graphical representation of the results of Example 8.

FIG. 24 is a graphical representation of the results of Example 8.

FIG. 25 is a graphical representation of the results of Example 8.

FIG. 26 is a graphical representation of the results of Example 8.

FIG. 27 is a graphical representation of the results of Example 8.

FIG. 28 is a graphical representation of the results of Example 8.

FIG. 29 is a graphical representation of the results of Example 8.

FIG. 30 is a graphical representation of the results of Example 9.

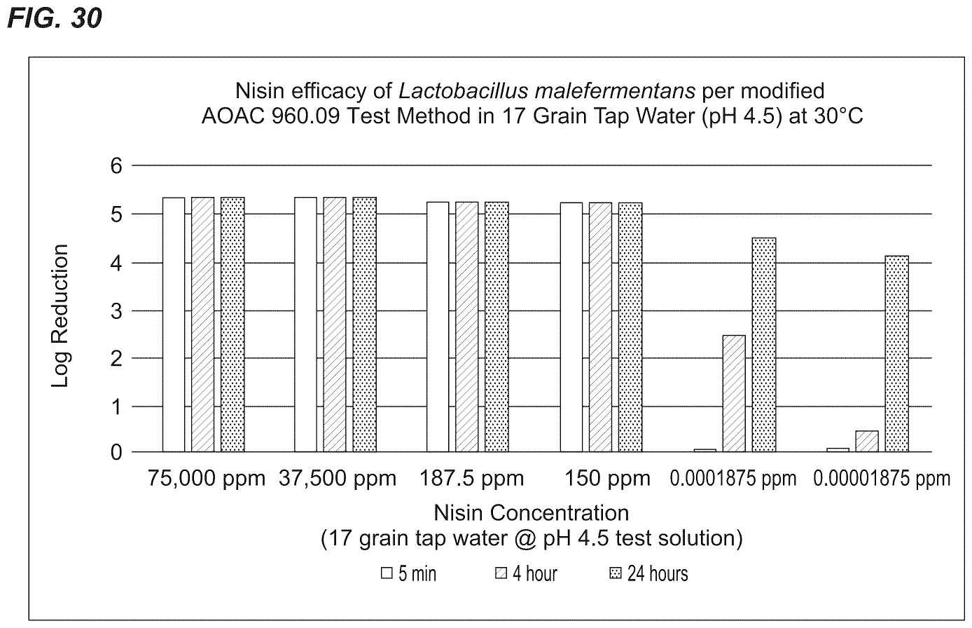

### DETAILED DESCRIPTION

As used herein, weight percent (wt. %), percent by weight, % by weight, and the like are synonyms that refer to the concentration of a substance as the weight of that substance divided by the total weight of the composition and multiplied by 100.

As used herein, the term “about” modifying the quantity of an ingredient in the compositions of the disclosure or employed in the methods of the disclosure refers to variation in the numerical quantity that can occur, for example, through typical measuring and liquid handling procedures used for making use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about,” the claims include equivalents to the quantities. The term “about” typically allows for a variation within =5% of the stated percent value. For example, if “about 10%” is used, the term “about” typically allows for a variation of about 5% of the stated percent value, 10%. Thus, “about 10%” may cover a variation of +0.5%.

As used herein, the terms “substantially free,” “substantially free of,” and “free of” of a particular substance means that the compositions of the instant specification contain less than 0.5 wt. % of the recited substance. When referring to “substantially free,” “substantially free of,” and “free of” it is intended that the substance is not intentionally added to the compositions. The term “essentially free” of a particular substance means that the compositions of the instant specification contain less than 0.1 wt. % of the recited substance. When referring to “essentially free” it is intended that the substance is not intentionally added to the compositions. The term “essentially completely free” of a particular substance means that the compositions of the instant specification contain less than 0.01 wt. % of the recited substance. When referring to “essentially completely free” it is intended that the substance is not intentionally added to the compositions. The term “completely free” of a particular substance means that the compositions of the instant specification contain less than 0.001 wt. % of the recited substance. When referring to “completely free” it is intended that the substance is not intentionally added to the compositions. Use of the term “completely free” allows for trace amounts of that substance to be included in compositions because they are present in another substance in the composition. However, it is recognized that only trace or de minimus amounts of a substance will be allowed when the composition is said to be “completely free” of that substance.

As used herein, the term “consisting essentially of” in reference to a composition refers to the listed ingredients and does not include additional ingredients that, if present, would affect the microbial reduction and/or control ability of the composition. The term “consisting essentially of” may also refer to a component of the composition. For instance, a composition for reducing and/or controlling microbial population may consist essentially of a compound or compounds and would not include any other ingredients that would affect the effectiveness of the composition at reducing or controlling microbial populations. As used herein, the term “consisting essentially of” in reference to a method of reducing or controlling microbial populations refers to the listed steps and does not include additional steps (or ingredients if a composition is included in the method) that, if present, would affect the microbial reduction or controlling ability of the method.

As used herein, the term “consisting essentially of” in reference to a composition refers to the listed ingredients and does not include additional ingredients that, if present, would affect the cleaning or sanitizing ability of the composition. The term “consisting essentially of” may also refer to a component of the composition. For instance, a composition for cleaning and/or sanitizing an ethanol fermentation system may consist essentially of a compound or compounds and would not include any other ingredients that would affect the effectiveness of the composition. As used herein, the term “consisting essentially of” in reference to a method of cleaning or sanitizing an ethanol fermentation system refers to the listed steps and does not include additional steps (or ingredients if a composition is included in the method) that, if present, would affect the cleaning or sanitizing efficacy of the method.

As used herein, the term “consisting of” in reference to a composition refers to the listed ingredients and does not include additional ingredients. The term “consisting of” explicitly excludes any additional components from the listed components. When “consisting of” is used in the preamble of a composition claim, the claim is closed off to any unrecited components. When “consisting of” is used in a clause of a composition claim, that clause is closed off to any unrecited components or steps. As used herein, the term “consisting of” in reference to a method refers to the listed steps and does not include additional steps. The term “consisting of” explicitly excludes any additional steps (or ingredients if a composition is included in the method) from the listed steps of a method claim. When “consisting of” is used in the preamble of a method claim, the claim is closed off to any unrecited steps. When “consisting of” is used in a clause of a method claim, that clause is closed off to any unrecited components or steps.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5. Ethanol production converts a biomass, such as corn, or other high-carbohydrate material, e.g., rice, nuts, wheat berries, sugar cane, and sugar beets, into ethanol. The process typically begins with grinding or milling the biomass, creating an aqueous slurry and often heating it to release fermentable sugars, followed by fermentation, where yeast converts these sugars into ethanol and carbon dioxide. After fermentation, the mixture may be distilled to separate the ethanol from other components, resulting in a high-purity alcohol. The byproducts of this process, such as spent grains, can be utilized for animal feed, enhancing overall resource efficiency.

As used herein, a “liquid” describes a state of matter that is flowable. A liquid can refer to a substance with a relatively low viscosity, such as water, but can also refer to a substance with a high viscosity, such as corn mash. A liquid can also refer to a substance that has components mixed throughout. For example, corn mash maybe comprise water and corn material and would be considered a liquid within the scope of the present disclosure.

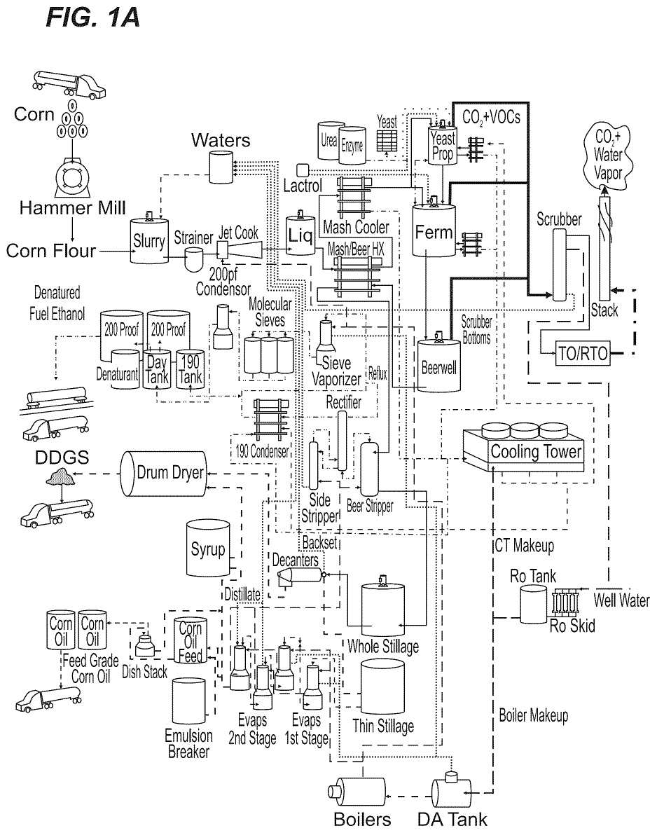

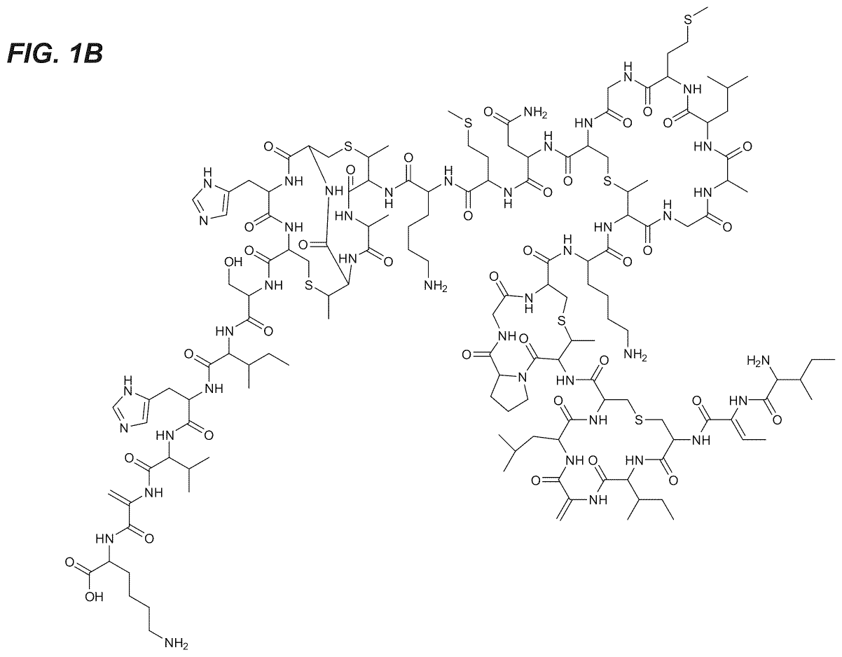

An ethanol fermentation system comprises many steps required to take a high-carbohydrate raw material and produce ethanol. An example of an ethanol fermentation system is shown in FIG. 1A. Microbial overgrowth is a concern at almost all stages of the ethanol fermentation process because microbial overgrowth in one step can lead to further proliferation as the intermediate products of ethanol production are moved throughout the system. For example, a biomass may be placed into a slurry tank with water, yeast, and other fermentation additives to begin the fermentation process. If unwanted bacteria are growing in the slurry tank and left unchecked, the bacteria may continue to proliferate as the contents of the slurry tank are passed through the ethanol system, which can reduce overall ethanol concentration and rate of ethanol production as the yeast compete with the bacteria for nutrients and may result in the production of unwanted byproducts and/or spoilage due to the bacterial overgrowth. Proliferation of unwanted bacteria can also cause incomplete fermentation that produces atypical ratios in sugar, ethanol, and water content. These imbalances can lead to problems with the fermentation equipment, such as sugars in distillation and evaporation that can burn on and plug equipment. Additionally, since ethanol production systems typically do not have wastewater streams and byproducts are recycled back to the beginning of the system, reuse of contaminated water sources may introduce unwanted bacteria and bacterial byproducts back into the system.

Many types of bacteria can proliferate in an ethanol production system, however, lactic acid- and acetic acid-producing bacteria pose particular challenges to ethanol production because these bacteria are naturally occurring and endemic. Additionally, these forms of bacteria produce organic acids, which lower the pH in the ethanol production system and can negatively impact yeast health. The present disclosure contemplates microbial population control for lactic acid-producing bacteria, acetic acid-producing bacteria, and other forms of bacteria.

Conventional solutions to address bacterial overgrowth of microorganisms in ethanol production often rely on the use of antibiotics. However, public concern regarding antibiotic resistance has made antibiotics a less favorable option for controlling bacterial overgrowth.

Additionally, industrial fermentation operations for making ethanol are constrained by strict standards for waste disposal. For example, most biofuel fermentation operations are not permitted to generate any waste streams and do not have water treatment systems. Therefore, the only way for material to leave an ethanol production plant is through steam, product streams (e.g., ethanol), or by-product streams (e.g., fermentation solids that are converted into another product such as animal feed, corn oil, or syrup). The by-product streams of ethanol production almost always must be Generally Recognized As Safe (“GRAS”) under US Food and Drug Administration guidelines in order to be used as food products for humans or animals. For these reasons, it may also be beneficial to reduce antibiotic use due to concerns with antibiotics ending up in by-product streams, such as animal feed.

In addition to microbial overgrowth, other forms of soiling within an ethanol production system can also lead to problems. Ethanol production equipment may become soiled with organic residues, biofilms, inorganic deposits, and particulate matter, which can lead to lower efficiency of heat transfer, harborages for microbial populations, and foulant accumulation. In order to maintain efficient ethanol production, these soils must be regularly removed from the ethanol production facility in a manner that produces GRAS by-product streams.

The present disclosure addresses these concerns by using compositions and methods to control microbial populations at multiple stages of an ethanol production process.

The present disclosure also addresses these concerns by using compositions and methods to control microbial populations at multiple stages of an ethanol production process, which thereby increases a rate of ethanol production and/or reduces an ethanol production time.

The present disclosure also addresses these concerns by using compositions and methods to clean, sanitize, and remove soil from the ethanol production equipment.

The present disclosure contemplates use in fermentation systems for production of carbohydrate ethanol, cellulosic ethanol, or combinations thereof.

Carbohydrate ethanol, often called starch- or sugar-based ethanol, is made from plant materials that already contain readily accessible carbohydrates. Common feedstock used for carbohydrate ethanol include corn, sugarcane, sugar beets, and other high-starch or high-sugar crops, such as those described herein. In this process, the carbohydrates are converted by breaking down starches into simple sugars, which are then fermented by yeast to produce ethanol.

Cellulosic ethanol is made from cellulose. Cellulose is the tough, fibrous material that makes up the cell walls of plants. Cellulose for fermentation is derived from non-edible biomass including but not limited to corn stover (e.g., stalks, leaves, husks, cobs), switch grass, miscanthus, other perennial grasses, wheat straw, forestry waste (e.g., wood chips, sawdust, tree tops, branches), or paper waste. Because cellulose is difficult for enzymes to break down, production involves pretreatment to open the fibers, enzymatic hydrolysis to release sugars, and fermentation to convert those sugars into ethanol.

In some examples, the present disclosure is directed to reducing and/or controlling microbial populations in carbohydrate ethanol production. In some examples, the present disclosure is only used in carbohydrate ethanol production. In some examples, the present disclosure is not used in carbohydrate ethanol production.

In some examples, the present disclosure is directed to reducing and/or controlling microbial populations in cellulosic ethanol production. In some examples, the present disclosure is only used in cellulosic ethanol production. In some examples, the present disclosure is not used in cellulosic ethanol production.

### Compositions

In some examples, a composition for reducing microbial populations in an ethanol production system comprises nisin. Nisin is a bacteriocin and lantibiotic that is produced by certain strains of Lactococcus lactis. Without wishing to be bound by theory, it is believed that nisin provides antimicrobial effects by binding to bacterial cell membranes and disrupting their integrity, often targeting lipid II, a key molecule in cell wall synthesis. It is believed that this action inhibits cell wall production and causes pore formation in the cell membrane, leading to leakage of essential ions and cell death. Nisin is particularly effective at reducing the population of gram-positive bacteria, such as Lactobacillus. Nisin has the molecular formula C₁₄₃H₂₃₀N₄₂O₃₇S₇ and its structure is shown in FIG. 1B.

The present disclosure contemplates using nisin in multiple ways in an ethanol production system. Nisin may be used in its pure form in which pure nisin is applied directly to the ethanol production system. Pure nisin may also be combined with other components to form a composition that is applied to the ethanol production system. Nisin may also be used as part of a nisin salt blend that is applied directly to the ethanol production system. A nisin salt blend may also be combined with other components to form a composition that is applied to the ethanol production system. The present disclosure also contemplates using nisin as the only antimicrobial added to the ethanol production system or as the only antimicrobial in a composition applied to the ethanol production system. The present disclosure also contemplates using nisin in combination with one or more additional antimicrobials that are added to the ethanol production system. The present disclosure contemplates using nisin as part of a holistic system for reducing microbial populations and cleaning throughout multiple stages of an ethanol production system.

As used herein, “nisin” may refer to pure nisin, a nisin carrier blend, a nisin salt blend, or combinations thereof, as described herein, unless otherwise specified.

In some examples, pure nisin may be used. As used herein, “pure nisin” refers to nisin that it is not mixed with a salt, a carrier, or other component. A level of purity less than 100% for “pure nisin” refers to impurities that may be present due to manufacturing limitations, isolation limitations, or contamination, for example. “Pure nisin” also refers to nisin that does not comprise any other components that have been intentionally added to the nisin.

Pure nisin refers to nisin that may be 100% pure, 99% pure, 98% pure, 97% pure, 96% pure, 95% pure, 94% pure, 93% pure, 92% pure, 91% pure, or 90% pure. Pure nisin may refer to nisin that is 90% to 100% pure, 91% to 100% pure, 92% to 100% pure, 93% to 100% pure, 94% to 100% pure, 95% to 100% pure, 96% to 100% pure, 97% to 100% pure, 98% to 100% pure, 99% to 100% pure, 90% to 99% pure, 90% to 98% pure, 90% to 97% pure, 90% to 96% pure, 90% to 95% pure, 90% to 94% pure, 90% to 93% pure, 90% to 92% pure, or 90% to 91% pure.

In examples where pure nisin is used, pure nisin may be applied directly to the ethanol production system. Alternatively, pure nisin may be incorporated into a composition comprising other components described herein.

In some examples, pure nisin is not used and may be part of a nisin product. As used herein, “nisin product” can refer to any composition in which nisin and a filler, carrier, or salt is included. As described herein, this may comprise a nisin carrier blend and a nisin salt blend.

In some examples, nisin may be part of a blend with fillers or carriers, referred to herein as a nisin carrier blend. The fillers or carriers may be any suitable filler or carrier in the art including, but not limited to, sugar, starch, sodium bicarbonate, cellulose, urea, maltose, yeast extract, sodium sulfate, calcium sulfate, and combinations thereof.

In examples where a nisin carrier blend is used, the nisin carrier blend may comprise about 2 wt. % to about 99.99 wt. % of nisin relative to the weight of the nisin carrier blend. For example, if a nisin carrier blend comprises about 2 wt. % of nisin, then the nisin carrier blend comprises about 98 wt. % of carrier. The nisin carrier blend may comprise about 2 wt. % to about 99.9 wt. %, about 2 wt. % to about 99 wt. %, about 2 wt. % to about 95 wt. %, about 2 wt. % to about 90 wt. %, about 2 wt. % to about 85 wt. %, about 2 wt. % to about 80 wt. %, about 2 wt. % to about 75 wt. %, about 2 wt. % to about 70 wt. %, about 2 wt. % to about 65 wt. %, about 2 wt. % to about 60 wt. %, about 2 wt. % to about 55 wt. %, about 2 wt. % to about 50 wt. %, about 2 wt. % to about 45 wt. %, about 2 wt. % to about 40 wt. %, about 2 wt. % to about 35 wt. %, about 2 wt. % to about 30 wt. %, about 2 wt. % to about 25 wt. %, about 2 wt. % to about 20 wt. %, about 2 wt. % to about 15 wt. %, about 2 wt. % to about 10 wt. %, or about 2 wt. % to about 5 wt. %, about 5 wt. % to about 99.9 wt. %, about 10 wt. % to about 99.9 wt. %, about 15 wt. % to about 99.9 wt. %, about 20 wt. % to about 99.9 wt. %, about 25 wt. % to about 99.9 wt. %, about 30 wt. % to about 99.9 wt. %, about 35 wt. % to about 99.9 wt. %, about 40 wt. % to about 99.9 wt. %, about 45 wt. % to about 99.9 wt. %, about 50 wt. % to about 99.9 wt. %, about 55 wt. % to about 99.9 wt. %, about 60 wt. % to about 99.9 wt. %, about 65 wt. % to about 99.9 wt. %, about 70 wt. % to about 99.9 wt. %, about 75 wt. % to about 99.9 wt. %, about 80 wt. % to about 99.9 wt. %, about 85 wt. % to about 99.9 wt. %, about 90 wt. % to about 99.9 wt. %, about 95 wt. % to about 99.9 wt. %, or about 99 wt. % to about 99.9 wt. % of nisin relative to the weight of the nisin carrier blend.

In examples where a nisin carrier blend is used, the nisin carrier blend may comprise about 0.01 wt. % to about 99.9 wt. % of carrier relative to the weight of the nisin carrier blend. For example, if a nisin carrier blend comprises about 0.01 wt. % of carrier, then the nisin carrier blend comprises about 99.9.9 wt. % of nisin. The nisin carrier blend may comprise about 0.01 wt. % to about 99 wt. %, 0.01 wt. % to about 98 wt. %, about 0.01 wt. % to about 95 wt. %, about 0.01 wt. % to about 90 wt. %, about 0.01 wt. % to about 85 wt. %, about 0.01 wt. % to about 80 wt. %, about 0.01 wt. % to about 75 wt. %, about 0.01 wt. % to about 70 wt. %, about 0.01 wt. % to about 65 wt. %, about 0.01 wt. % to about 60 wt. %, about 0.01 wt. % to about 55 wt. %, about 0.01 wt. % to about 50 wt. %, about 0.01 wt. % to about 45 wt. %, about 0.01 wt. % to about 40 wt. %, about 0.01 wt. % to about 35 wt. %, about 0.01 wt. % to about 30 wt. %, about 0.01 wt. % to about 25 wt. %, about 0.01 wt. % to about 20 wt. %, about 0.01 wt. % to about 15 wt. %, about 0.01 wt. % to about 10 wt. %, about 0.01 wt. % to about 5 wt. %, about 5 wt. % to about 99.9 wt. %, about 10 wt. % to about 99.9 wt. %, about 15 wt. % to about 99.9 wt. %, about 20 wt. % to about 99.9 wt. %, about 25 wt. % to about 99.9 wt. %, about 30 wt. % to about 99.9 wt. %, about 35 wt. % to about 99.9 wt. %, about 40 wt. % to about 99.9 wt. %, about 45 wt. % to about 99.9 wt. %, about 50 wt. % to about 99.9 wt. %, about 55 wt. % to about 99.9 wt. %, about 60 wt. % to about 99.9 wt. %, about 65 wt. % to about 99.9 wt. %, about 70 wt. % to about 99.9 wt. %, about 75 wt. % to about 99.9 wt. %, about 80 wt. % to about 99.9 wt. %, about 85 wt. % to about 99.9 wt. %, about 90 wt. % to about 99.9 wt. %, about 95 wt. % to about 99.9 wt. %, or about 99 wt. % to about 99.9 wt. % of the carrier relative to the weight of the nisin carrier blend.

In some examples, nisin may be part of a nisin salt blend. As used herein, a nisin salt blend refers to a mixture of nisin and a salt. The nisin salt blend may comprise sodium, potassium, calcium, magnesium, ammonium, or combinations thereof. The nisin salt may also comprise chloride, carbonate, bicarbonate, sulfate, nitrate, hydroxide, and other suitable compounds for forming a salt, or combinations thereof. Some examples of salts to be blended with nisin include, but are not limited to, sodium chloride, sodium bicarbonate, sodium carbonate, potassium chloride, calcium chloride, calcium carbonate, magnesium sulfate, and combinations thereof. Some examples of nisin salt blends include, but are not limited to, nisin plus sodium chloride, nisin plus sodium bicarbonate, nisin plus sodium carbonate, nisin plus potassium chloride, nisin plus calcium chloride, nisin plus calcium carbonate, nisin plus magnesium sulfate, and combinations thereof.

In examples where a nisin salt blend is used, the nisin salt blend may comprise about 2 wt. % to about 99.99 wt. % of nisin relative to the weight of the nisin salt blend. For example, if a nisin salt blend comprises about 2 wt. % of nisin, then the nisin salt blend comprises about 98 wt. % of salt. The nisin salt blend may comprise about 2 wt. % to about 99.9 wt. %, about 2 wt. % to about 99 wt. %, about 2 wt. % to about 95 wt. %, about 2 wt. % to about 90 wt. %, about 2 wt. % to about 85 wt. %, about 2 wt. % to about 80 wt. %, about 2 wt. % to about 75 wt. %, about 2 wt. % to about 70 wt. %, about 2 wt. % to about 65 wt. %, about 2 wt. % to about 60 wt. %, about 2 wt. % to about 55 wt. %, about 2 wt. % to about 50 wt. %, about 2 wt. % to about 45 wt. %, about 2 wt. % to about 40 wt. %, about 2 wt. % to about 35 wt. %, about 2 wt. % to about 30 wt. %, about 2 wt. % to about 25 wt. %, about 2 wt. % to about 20 wt. %, about 2 wt. % to about 15 wt. %, about 2 wt. % to about 10 wt. %, or about 2 wt. % to about 5 wt. %, about 5 wt. % to about 99.9 wt. %, about 10 wt. % to about 99.9 wt. %, about 15 wt. % to about 99.9 wt. %, about 20 wt. % to about 99.9 wt. %, about 25 wt. % to about 99.9 wt. %, about 30 wt. % to about 99.9 wt. %, about 35 wt. % to about 99.9 wt. %, about 40 wt. % to about 99.9 wt. %, about 45 wt. % to about 99.9 wt. %, about 50 wt. % to about 99.9 wt. %, about 55 wt. % to about 99.9 wt. %, about 60 wt. % to about 99.9 wt. %, about 65 wt. % to about 99.9 wt. %, about 70 wt. % to about 99.9 wt. %, about 75 wt. % to about 99.9 wt. %, about 80 wt. % to about 99.9 wt. %, about 85 wt. % to about 99.9 wt. %, about 90 wt. % to about 99.9 wt. %, about 95 wt. % to about 99.9 wt. %, or about 99 wt. % to about 99.9 wt. % of nisin relative to the weight of the nisin salt.

In examples where a nisin salt blend is used, the nisin salt blend may comprise about 0.01 wt. % to about 99.9 wt. % of salt relative to the weight of the nisin salt blend. For example, if a nisin salt blend comprises about 0.01 wt. % of salt, then the nisin salt blend comprises about 99.99 wt. % of nisin. The nisin salt blend may comprise about 0.01 wt. % to about 99 wt. %, about 0.01 wt. % to about 98 wt. %, about 0.01 wt. % to about 95 wt. %, about 0.01 wt. % to about 90 wt. %, about 0.01 wt. % to about 85 wt. %, about 0.01 wt. % to about 80 wt. %, about 0.01 wt. % to about 75 wt. %, about 0.01 wt. % to about 70 wt. %, about 0.01 wt. % to about 65 wt. %, about 0.01 wt. % to about 60 wt. %, about 0.01 wt. % to about 55 wt. %, about 0.01 wt. % to about 50 wt. %, about 0.01 wt. % to about 45 wt. %, about 0.01 wt. % to about 40 wt. %, about 0.01 wt. % to about 35 wt. %, about 0.01 wt. % to about 30 wt. %, about 0.01 wt. % to about 25 wt. %, about 0.01 wt. % to about 20 wt. %, about 0.01 wt. % to about 15 wt. %, about 0.01 wt. % to about 10 wt. %, about 0.01 wt. % to about 5 wt. %, about 5 wt. % to about 99.9 wt. %, about 10 wt. % to about 99.9 wt. %, about 15 wt. % to about 99.9 wt. %, about 20 wt. % to about 99.9 wt. %, about 25 wt. % to about 99.9 wt. %, about 30 wt. % to about 99.9 wt. %, about 35 wt. % to about 99.9 wt. %, about 40 wt. % to about 99.9 wt. %, about 45 wt. % to about 99.9 wt. %, about 50 wt. % to about 99.9 wt. %, about 55 wt. % to about 99.9 wt. %, about 60 wt. % to about 99.9 wt. %, about 65 wt. % to about 99.9 wt. %, about 70 wt. % to about 99.9 wt. %, about 75 wt. % to about 99.9 wt. %, about 80 wt. % to about 99.9 wt. %, about 85 wt. % to about 99.9 wt. %, about 90 wt. % to about 99.9 wt. %, about 95 wt. % to about 99.9 wt. %, or about 99 wt. % to about 99.9 wt. % of the salt relative to the weight of the nisin salt blend.

In some examples, the concentration of nisin may be measured relative to the volume or weight of the particular step of the system that the nisin may be added to. For example, if the nisin is added to a mash tank or a fermentation tank, the concentration of the nisin may be measured relative to the volume of the mash in the mash tank or relative to the volume of the ferment in the fermentation tank.

In some examples, the concentration of nisin is measured relative to the liquid volume in the ethanol production vessel that the nisin is added to. In some examples, an amount of a nisin salt blend, a nisin carrier blend, or pure nisin added to the vessel is about 0.001 to about 60 kg per 10,000 gallons of liquid in the vessel. The amount of the nisin salt blend, the nisin carrier blend, or pure nisin per 10,000 gallons of liquid in the vessel may comprise about 0.001 kg to about 60 kg, about 0.005 kg to about 60 kg, about 0.01 kg to about 60 kg, about 0.05 kg to about 60 kg, about 0.1 kg to about 60 kg, about 0.5 kg to about 60 kg, about 1.0 kg to about 60 kg, about 5 kg to about 60 kg, about 10 kg to about 60 kg, about 15 kg to about 60 kg, about 20 kg to about 60 kg, about 25 kg to about 60 kg, about 30 kg to about 60 kg, about 35 kg to about 60 kg, about 40 kg to about 60 kg, about 45 kg to about 60 kg, about 50 kg to about 60 kg, or about 55 kg to about 60 kg.

Commercially available nisin compositions generally describe the amount of nisin in International Units (IU) per mg of composition. Nisin activity, expressed in IU/mg, refers to the amount of nisin required to inhibit growth of 1 bacterial cell in 1 milliliter of broth. In some examples, where a nisin salt blend or a nisin carrier blend is used, the nisin salt blend or the nisin carrier blend may comprise about 5 IU/mg to about 41,000 IU/mg. The nisin salt blend or the nisin carrier blend may comprise about 5 to about 41,000 IU/mg, about 50 to about 41,000 IU/mg, about 100 to about 41,000 IU/mg, about 200 to about 41,000 IU/mg, about 300 to about 41,000 IU/mg, about 400 to about 41,000 IU/mg, about 500 to about 41,000 IU/mg, about 600 to about 41,000 IU/mg, about 700 to about 41,000 IU/mg, about 800 to about 41,000 IU/mg, about 900 to about 41,000 IU/mg, about 1,000 to about 41,000 IU/mg, about 2,000 to about 41,000 IU/mg, about 3,000 to about 41,000 IU/mg, about 4,000 to about 41,000 IU/mg, about 5,000 to about 41,000 IU/mg, about 6,000 to about 41,000 IU/mg, about 7,000 to about 41,000 IU/mg, about 8,000 to about 41,000 IU/mg, about 9,000 to about 41,000 IU/mg, about 10,000 to about 41,000 IU/mg, about 12,000 to about 41,000 IU/mg, about 14,000 to about 41,000 IU/mg, about 16,000 to about 41,000 IU/mg, about 18,000 to about 41,000 IU/mg, about 20,000 to about 41,000 IU/mg, about 22,000 to about 41,000 IU/mg, about 24,000 to about 41,000 IU/mg, about 26,000 to about 41,000 IU/mg, about 28,000 to about 41,000 IU/mg, about 30,000 to about 41,000 IU/mg, about 32,000 to about 41,000 IU/mg, about 34,000 to about 41,000 IU/mg, about 36,000 to about 41,000 IU/mg, about 38,000 to about 41,000 IU/mg, or about 40,000 to about 41,000 IU/mg.

Pure nisin or a nisin product (such as a nisin carrier blend and/or a nisin salt blend) may also be added to a fermentation system as described herein in terms of parts per million (ppm). As used herein, “ppm” or “parts per million” refers to the mass of the nisin product (e.g., pure nisin, nisin carrier blend, and/or nisin salt blend) divided by the mass of the total solution that the nisin product is added to. In examples where ppm is measured relative to an at scale fermentation system (e.g., a large scale commercial fermentation system), ppm is calculated in accordance with Equation 1:

$$Nisin ( ppm ) = \frac{Mass of Nisin Product ( kg ) * X}{\begin{matrix}Fermentation Fill Volume ( gallons ) * \\ 3.785 \frac{L}{gal} * 0.75 kg / L\end{matrix}} \qquad Eqn 1$$

Equation 1 uses 3.785 gallons as a standard unit conversion and the total contents of a standard fermentation reaction is assumed to have a density of 0.75 kg/L based on industry standards. To calculate the ppm of nisin alone (i.e., nisin without considering a carrier or salt), the mass of the nisin product in filler or salt form can be multiplied by X, which represents the percentage of nisin product that is nisin. The percentage of nisin product that comprises nisin can be calculated based on the ratio of nisin to carrier or salt in the nisin product. For example, for a nisin product (e.g., nisin carrier blend or nisin salt blend) that is 60% nisin, X is 0.6.

In examples where ppm is measured, for example, in a laboratory setting where the reaction conditions are more easily controlled, ppm can be measured in accordance with Equation 2:

$$Nisin ( ppm ) = ( ( ( \frac{Mass of Nisin Product ( g ) * X}{Mass of Total Solution ( g )} ) \times 100 ) \times 10 , 000 )$$

Equation 2 measures the nisin ppm in terms of mass of nisin product and mass of total solution in grams as a lab setting likely would measure quantities of product in grams, rather large quantities, such as kilograms. However, the masses in Equation 2 can be measured in kg or other suitable unit of measurement in the art. Equation 2 does not require calculations based on density as the total mass of the solution can be easily measured. In contrast, measuring total mass of a solution in a fermentation system setting is more complicated when, for example, the fermentation vessel may be a tank with a volume of 100,000-900,000 gallons or larger. To calculate the ppm of nisin alone (i.e., nisin without considering a carrier or salt), the mass of the nisin product in filler or salt form can be multiplied by X, which represents the percentage of nisin product that is nisin. The percentage of nisin product that comprises nisin can be calculated based on the ratio of nisin to carrier or salt in the nisin product. For example, for a nisin product (e.g., nisin carrier blend or nisin salt blend) that is 60% nisin, X is 0.6.

As described herein, ppm of nisin refers to the amount of nisin peptide added to a solution. For example, there may be 100 ppm of a nisin salt blend in a solution (such as a volume of liquid in a fermentation vessel or other test liquid), where 20 ppm are nisin (i.e., the peptide) and where 80 ppm are a salt. Unless specified otherwise herein, ppm of nisin refers to the amount of nisin itself.

In some examples, nisin may be added to the fermentation system in quantities of about 0.0001 ppm to about 75,000 ppm relative to the volume of liquid in the fermentation vessel. In some examples, nisin may be added to the fermentation system in quantities of about 0.0001 ppm to about 70,000 ppm, about 0.0001 ppm to about 65,000 ppm, about 0.0001 ppm to about 60,000 ppm, about 0.0001 ppm to about 55,000 ppm, about 0.0001 ppm to about 50,000 ppm, about 0.0001 ppm to about 45,000 ppm, about 0.0001 ppm to about 40,000 ppm, about 0.0001 ppm to about 35,000 ppm, about 0.0001 ppm to about 30,000 ppm, about 0.0001 ppm to about 25,000 ppm, about 0.0001 ppm to about 20,000 ppm, about 0.0001 ppm to about 15,000 ppm, about 0.0001 ppm to about 10,000 ppm, about 0.0001 ppm to about 5,000 ppm, about 0.0001 ppm to about 1,000 ppm, about 0.0001 ppm to about 750 ppm, about 0.0001 ppm to about 500 ppm, about 0.0001 ppm to about 400 ppm, about 0.0001 ppm to about 300 ppm, about 0.0001 ppm to about 250 ppm, about 0.0001 ppm to about 200 ppm, about 0.0001 ppm to about 150 ppm, about 0.0001 ppm to about 100 ppm, about 0.0001 ppm to about 75 ppm, about 0.0001 ppm to about 50 ppm, about 0.0001 ppm to about 25 ppm, about 0.0001 ppm to about 20 ppm, about 0.0001 ppm to about 15 ppm, about 0.0001 ppm to about 10 ppm, about 0.0001 ppm to about 9 ppm, about 0.0001 ppm to about 8 ppm, about 0.0001 ppm to about 7 ppm, about 0.0001 ppm to about 6 ppm, about 0.0001 ppm to about 5 ppm, about 0.0001 ppm to about 4.5 ppm, about 0.0001 ppm to about 4 ppm, about 0.0001 ppm to about 3.5 ppm, about 0.0001 ppm to about 3 ppm, about 0.0001 ppm to about 2.5 ppm, about 0.0001 ppm to about 2 ppm, about 0.0001 ppm to about 1.5 ppm, about 0.0001 ppm to about 1 ppm, about 0.0001 ppm to about 0.5 ppm, about 0.0001 ppm to about 0.1 ppm, about 0.0001 ppm to about 0.05 ppm, about 0.0001 ppm to about 0.01 ppm, about 0.001 ppm to about 75,000 ppm, about 0.01 ppm to about 75,000 ppm, about 0.05 ppm to about 75,000 ppm, about 0.1 ppm to about 75,000 ppm, about 0.5 ppm to about 75,000 ppm, 1 ppm to about 75,000 ppm, about 1.5 ppm to about 75,000 ppm, about 2 ppm to about 75,000 ppm, 2.5 ppm to about 75,000 ppm, about 3 ppm to about 75,000 ppm, about 3.5 ppm to about 75,000 ppm, about 4 ppm to about 75,000 ppm, about 4.5 ppm to about 75,000 ppm, 5 ppm to about 75,000 ppm, about 6 ppm to about 75,000 ppm, about 7 ppm to about 75,000 ppm, about 8 ppm to about 75,000 ppm, about 9 ppm to about 75,000 ppm, 10 ppm to about 75,000 ppm, 15 ppm to about 75,000 ppm, 20 ppm to about 75,000 ppm, 25 ppm to about 75,000 ppm, 50 ppm to about 75,000 ppm, 75 ppm to about 75,000 ppm, 100 ppm to about 75,000 ppm, 150 ppm to about 75,000 ppm, 200 ppm to about 75,000 ppm, 250 ppm to about 75,000 ppm, 300 ppm to about 75,000 ppm, 400 ppm to about 75,000 ppm, 500 ppm to about 75,000 ppm, 750 ppm to about 75,000 ppm, 1,000 ppm to about 75,000 ppm, 5,000 ppm to about 75,000 ppm, 10,000 ppm to about 75,000 ppm, 15,000 ppm to about 75,000 ppm, 20,000 ppm to about 75,000 ppm, 25,000 ppm to about 75,000 ppm, 30,000 ppm to about 75,000 ppm, 35,000 ppm to about 75,000 ppm, 40,000 ppm to about 75,000 ppm, 45,000 ppm to about 75,000 ppm, 50,000 ppm to about 75,000 ppm, 55,000 ppm to about 75,000 ppm, 60,000 ppm to about 75,000 ppm, 65,000 ppm to about 75,000 ppm, or about 70,000 ppm to about 75,000 ppm.

Because nisin can be used in a wide variety of manners in the ethanol production system, the quantity of nisin to be used can vary extensively. For example, the quantity of nisin to be used may depend upon the form of nisin. A nisin salt blend that comprises 5 wt. % nisin and 95 wt. % sodium chloride may need to be added in a larger amount compared to a nisin salt blend that comprises 80 wt. % nisin and 20 wt. % sodium chloride because the concentrations of nisin relative to the nisin salt blend vary extensively. The present disclosure contemplates a wide range of nisin concentrations that are dependent upon the form of the nisin, the purity of the nisin, where the nisin is added in the system, the volume of the intermediate products in the vessel the nisin is added to, and the other components that may be present in the composition.

Multiple methods of measuring nisin are described herein (e.g., wt. %, IU/mg, and ppm). When selecting nisin concentration for a fermentation system, in some examples, it is beneficial to consider both the concentration (wt. % or ppm) and activity of nisin (IU/mg). Nisin concentration refers to the amount of nisin protein present in a solution, which may be composed of water, corn mash, or other fermentation broths. For example, 5 wt. % nisin refers to 5 wt. % of nisin protein relative to the volume of the solution the nisin is added to. Nisin product concentration refers to the amount of nisin in combination with a salt or a carrier in a solution. Alternatively, nisin activity refers to the efficacy by which nisin controls bacteria competing with yeast during fermentation. Nisin activity is typically related to a dose-response, wherein the efficacy is determined for a given amount of protein. A preparation of nisin may have varying degrees of efficacy depending on the conformational state of the protein. Thus, an assay rating is identified for every batch to quantify the anticipated efficacy outcome, as quantified in units of IU/mg.

The activity of nisin can be measured using a suspension-based microbiological test assay under controlled conditions to evaluate a reduction in Lactobacillus by comparing nisin of a known activity to that of a new, alternate or unknown nisin activity level. For example, manufacturers of nisin may provide a known activity for nisin. Following treatment of Lactobacillus with the nisin preparation(s), test samples can be plated onto appropriate agar medium and incubated for recovery of any surviving organisms. The results from nisin-treated samples are compared to an untreated control to determine the reduction value, thereby assessing the performance across each nisin sample. This then can be used to measure the activity of nisin.

When considering concentration of nisin, it is important to consider the activity of nisin as well. For example, 50 ppm of nisin with an activity of about 1,000 IU/mg is less active than 50 ppm of nisin with an activity of about 30,000 IU/mg. As described above, nisin may have varying degrees of efficacy given that it is a protein subject to conformational changes—therefore, the activity of nisin in the measured concentration is needed to properly evaluate the amount of active nisin is added (i.e., the amount of nisin that is capable of producing the desired reduction and/or control of microbial populations). For example, a certain quantity (e.g., ppm) of nisin with an activity of about 30,000 IU/mg has an activity that is about 30-times greater than the same quantity of nisin with an activity of about 1,000 IU/mg, though there may be variations due to unavoidable variability in product consistency. Nisin with a higher activity has the potential to have increased effects on controlling microbial populations. Therefore, when referring to concentration of nisin, it is inadequate to consider only the concentration of nisin in terms of ppm or wt. % as these values do not indicate the activity of the nisin.

When nisin concentrations in the field are referred to only with wt. % or ppm and no indication of nisin activity (i.e., IU/mg), this is insufficient information to determine whether the concentration of nisin will have the desired impact on reducing and/or controlling microbial populations. For example, a recitation of 5 ppm nisin without specifying the activity of nisin does not provide a skilled artisan with sufficient information to evaluate whether the concentration of nisin will have the desired impact on reducing and/or controlling microbial populations.

While the present application describes large ranges of carrier or salt in nisin carrier blends and nisin salt blends, respectively, in some examples it may be advantageous to have concentrations of nisin with lower quantities of carrier and/or salt. For example, when nisin is in a nisin salt blend with sodium chloride, it may be beneficial to select a higher concentration of nisin relative to sodium chloride as sodium ion concentration in fermentation operations may negatively impact fermentation. Too much sodium can result in damage to yeast, alter final ethanol products and concentration, reduce efficiency of fermentation, and/or produce undesirable byproducts.

The present disclosure contemplates adding nisin to the ethanol production system without additional antimicrobial agents.

The present disclosure also contemplates forming a composition by adding other components, including one or more antimicrobial agents, in combination with nisin to the ethanol production system.

The present disclosure also contemplates using one or more antimicrobial agents in the ethanol production system without using nisin. For example, the one or more antimicrobials described herein can be added to the ethanol production system to reduce and/or control microbial populations without adding nisin. In some examples, the composition comprises one or more antimicrobial agents in addition to, or in the alternative of, nisin. Such antimicrobial agents may be peptide AA230, polylysine, tetraacetylethylenediamine (TAED), phosphonate, hop acid or hop acid extract, thymol, sodium bisulfite, organic acids, or combinations thereof.

AA230 is a peptide derived from arenicin-3 that demonstrates activity against gram negative bacteria, such as acetic acid-producing bacteria. Polylysine also demonstrates activity against gram negative bacteria. AA230 and/or polylysine may be useful in combination with nisin to provide reduction against both gram negative and gram positive bacteria.

In some examples, the composition may be free of AA230. In some examples, the composition may be essentially free of AA230. In some examples, the composition may be essentially completely free of AA230. In some examples, the composition may be completely free of AA230.

In some examples, the composition may be free of polylysine. In some examples, the composition may be essentially free of polylysine. In some examples, the composition may be essentially completely free of polylysine. In some examples, the composition may be completely free of polylysine.

In some examples, the composition may be free of TAED. In some examples, the composition may be essentially free of TAED. In some examples, the composition may be essentially completely free of TAED. In some examples, the composition may be completely free of TAED.

In some examples, the composition may be free of phosphonate. In some examples, the composition may be essentially free of phosphonate. In some examples, the composition may be essentially completely free of phosphonate. In some examples, the composition may be completely free of phosphonate.

In some examples, the composition may be free of thymol. In some examples, the composition may be essentially free of thymol. In some examples, the composition may be essentially completely free of thymol. In some examples, the composition may be completely free of thymol.

In some examples, the composition may be free of sodium bisulfite. In some examples, the composition may be essentially free of sodium bisulfite. In some examples, the composition may be essentially completely free of sodium bisulfite. In some examples, the composition may be completely free of sodium bisulfite.

In some examples, the composition may be free of organic acids. In some examples, the composition may be essentially free of organic acids. In some examples, the composition may be essentially completely free of organic acids. In some examples, the composition may be completely free of organic acids.

Hop acid (also referred to as hop acid extract) can be extracted from hops and used as an antimicrobial agent. Hop acids comprise a phenol group that provides antimicrobial activity against gram negative bacteria, particularly acetic acid-producing bacteria. The general term “hop acid” can refer to alpha acids, isomerized acids, rho isomerized alpha acids, tetra isomerized alpha acids, hex isomerized alpha acids, beta acid compounds, hop leaf, derivatives thereof, and combinations thereof.

Hop acids are generally provided in liquid form which may not be useful in some of the methods described herein, as the addition of a liquid composition would require the use of a separate dosing system. For example, adding a liquid to a fermentation system requires one or more pumps to transfer the liquid to the fermentation system or manually adding the liquid to the system, which can be time consuming and difficult. In contrast, a powdered/solid product can be more easily added to a fermentation system. In some examples, the methods described herein do not comprise the use of a dosing system for adding a liquid composition. In some examples, the systems, compositions, and methods described herein do not comprise liquids or the addition of liquids to the system.

In some examples, the composition may be free of hop acid. In some examples, the composition may be essentially free of hop acid. In some examples, the composition may be essentially completely free of hop acid. In some examples, the composition may be completely free of hop acid.

The present disclosure contemplates compositions in which nisin is effective as the only component added that is intended to reduce or control microbial populations and other components that are intended to reduce or control microbial populations are excluded. As described herein for the present applications, salt is not considered to be a component intended to reduce or control microbial populations.

Not only can nisin be used as the only component added that is intended to reduce or control microbial populations, large quantities of nisin are not needed to demonstrate efficacy. The present application describes and demonstrates the efficacy of large ranges of nisin concentration and activity, but relatively low concentrations of nisin are sufficient to demonstrate efficacy. In some examples, nisin is the only component added intended to reduce or control microbial populations and is present in the composition in quantities of about 0.001 ppm to about 1 ppm, about 0.01 ppm to about 1 ppm, about 0.1 to about 1 ppm, 0.5 ppm to about 1 ppm, about 0.001 ppm to about 0.5 ppm, about 0.001 ppm to about 0.1 ppm, or about 0.001 ppm to about 0.01 ppm with activity of about 30,000 IU/mg. The ability to effectively reduce or control microbial populations in a fermentation system with only nisin in the described quantities and activity represents an improvement in the field. Higher concentrations of nisin (e.g., 5 ppm or higher) demonstrate efficacy at reducing and/or controlling microbial populations as described herein, but it is a further development to demonstrate efficacy with lower concentrations of nisin and as the only component for reducing and/or controlling microbial populations.

In some examples, the composition may comprise one or more surfactants. The one or more surfactants may be selected from the group consisting of sodium xylene sulfonate, polysorbate 20, polysorbate 40, polysorbate 60, polysorbate 80, amine oxides, and combinations thereof. Examples of polysorbate surfactants that are commercially sold are TWEEN® 20, TWEEN® 40, TWEEN® 60, and TWEEN® 80. Use of additional surfactants is contemplated, including surfactants that are nonionic, anionic, cationic, zwitterionic, amphoteric, or combinations thereof.

In some examples, the composition may comprise one or more chelating agents. The chelating agents may be selected from the group consisting of EDTA, DTPA, HEDTA, HEDP, MGDA, and combinations thereof. The chelating agents may act in the composition to bind metal ions present in an ethanol production system to prevent and remove scaling and deposits that may form in various components of the ethanol production system.

In some examples, the composition may comprise one or more carriers. The carrier may be a solid carrier, an aqueous carrier, or other liquid carrier. The carrier used for the composition depends on whether the composition is in the form of a solid, such as a dry powder, or if the composition is a liquid, both of which are disclosed herein. In some examples, the composition is a dry powder, thereby the adding the composition to the fermentation system does not require a dosing system as described herein.

The carrier may comprise sodium chloride, sugar, starch, sodium bicarbonate, cellulose, urea, maltose, yeast extract, sodium sulfate, calcium sulfate, and combinations thereof. The carrier may also be alcohol-based, alcohol, water-based, water, or combinations thereof.

In some examples, the composition may comprise one or more additives selected from the group consisting of anti-caking agents, disintegrants, solubilizers, dyes, dispersants, preservatives, humectants, pH buffers, suspension agents, and combinations thereof.

In some examples, the composition is free of sodium lactate. In some examples, the composition is essentially free of sodium lactate. In some examples, the composition is completely free of sodium lactate.

In some examples, the composition is free of sodium acetate. In some examples, the composition is essentially free of sodium acetate. In some examples, the composition is complete free of sodium acetate.

In some examples, the composition is free of sodium lactate and sodium acetate. In some examples, the composition is essentially free of sodium lactate and sodium acetate. In some examples, the composition is completely free of sodium lactate and sodium acetate.

In some examples, the composition is free of biocides. In some examples, the composition is essentially free of biocides. In some examples, the composition is completely free of biocides. In some examples, the composition is free of, essentially free of, or completely free of biocides selected from the group consisting of 2.2-dibromo-3-nitrilopropionamide, methylene bisthiocyan ate, 2-(thiocyanomethyl-thio)benzothiazole, cyanodithiocarbimate salt, N-methyldithiocarbamate salt, polyoxyethylene(dimethyliminio)-ethylene (dimeth yliminio)ethylene dichloride, tetrakis(hydroxymethyl)phosphoniumsulfate, 1,1,1-tris(hydroxymethyl)nitromethane, glutaraldehyde, 1,5-pentanedial, alkylbenzyl ammonium chloride, 2-bromo-2-nitro-propane-1,3-diol, didecyl dim ethyl ammonium chloride, dimethyldithiocarbamate salt, dodecylguanidine hydrochloride, 1,2-benzisothiazoline-3-one, 5-chloro-2-methyl-4-isothiazolin-3-one, 2-methyl-4-isothiazoline-3-one, n-octyl isothiazolinone, dichloro-n-octylisothiazolinone, bromonitrostyrene, or tetrahydro-3, 5-dimethyl-2H-1,3,5-thiadiazine-2-thione, and combinations thereof. In some examples, the composition is free of, essentially free of, or completely free of biocides, including, but not limited to peracetic acid, sodium hypochlorite, alcohols, chlorine-containing compounds, quaternary ammonium compounds, acids, and other biocides known in the field.

In some examples, the compositions described herein are yeast safe or yeast compatible. As used herein, “yeast safe” and “yeast compatible” refer to compositions that do not significantly damage the yeast present in the ethanol production system that are necessary for fermentation. In some examples, the compositions described herein have antimicrobial properties that target unwanted bacteria without significantly damaging the yeast necessary for fermentation.

The compositions described herein may have a wide range of pHs depending on the components of the composition and method and location of use in the ethanol production system. For example, when the composition is in a liquid form, the composition alone may have a pH of about 2 to about 10, about 2 to about 9, about 2 to about 8, about 2 to about 7, about 2 to about 6, about 2 to about 5, about 2 to about 4, about 2 to about 3, about 3 to about 10, about 4 to about 10, about 5 to about 10, about 6 to about 10, about 7 to about 10, about 8 to about 10, or about 9 to about 10.

In some examples, the desired pH of the composition is measured relative to the part of the ethanol production system that the composition is added to. For example, when the composition is added to the mash, the pH of the mixture of the mash and the composition may be about 2 to about 8, about 2 to about 7, about 2 to about 6, about 2 to about 5, about 2 to about 4, about 2 to about 3, about 3 to about 8, about 4 to about 8, about 5 to about 8, about 6 to about 8, or about 7 to about 8. When the composition is added to other parts of the ethanol production system, the pH may depend upon the desired pH for the particular step of the ethanol production process. For example, it may be desirable for the pH in the fermentation tank to be acidic, and thus the pH of the ferment and composition mixture in the fermentation tank may be acidic.

### Methods

The compositions and methods described herein may be used in an ethanol production system in many ways. The example ethanol production system shown in FIG. 1A comprises many different steps and vessels that are used in ethanol production.

In some examples, a composition may be added to an ethanol production system. The composition may comprise any of the components described herein, including nisin with or without other components. The composition may be added to various parts of the ethanol production system.

The compositions described herein may be added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof. The present disclosure also contemplates adding the compositions described herein to other parts of the ethanol production system that may be susceptible to microbial population overgrowth.

The present disclosure also contemplates a method of increasing a rate of ethanol production in an ethanol production system. In some examples, adding a composition described herein to an ethanol production system increases an overall rate of ethanol production, relative to a system under the same or similar conditions where the composition has not been added.

In some examples, the rate of ethanol production is increased about 0.1% to about 50%, about 0.1% to about 45%, about 0.1% to about 40%, about 0.1% to about 35%, about 0.1% to about 33%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, about 0.1% to about 1%, or about 0.1% to about 0.5%. In some examples, an amount the rate of ethanol production is increased depends on an initial amount of microbial population overgrowth prior to adding the nisin.

Without wishing to be bound by theory, it is believed that ethanol production systems having higher baseline lactic acid concentrations achieve larger increases in the rate of ethanol production upon the addition of nisin. This is likely due to the nisin slowing the proliferation and/or reducing the bacterial populations of the lactic acid producing bacteria within the ethanol production system.

The present disclosure also contemplates a method of decreasing an ethanol production time in an ethanol production system. In some examples, adding a composition described herein to an ethanol production system decreases an ethanol production time, relative to a system under the same or similar conditions where the composition has not been added.

As used herein, “ethanol production time” may refer to an amount of time between the beginning of an ethanol fermentation reaction and the point at which the average baseline ethanol concentration is reached for that particular fermentation operation without the addition of nisin. Each fermentation operation may have an average baseline ethanol concentration in which the fermentation operation is “complete” such that fermentation no longer occurs or has slowed down significantly, a majority of the sugar has been consumed, and the time required to continue the fermentation process outweighs the benefits of additional amounts of ethanol that may be produced. The final ethanol concentrations from an ethanol production system are averaged to reach the average baseline ethanol concentration for a particular fermentation operation in the ethanol production system. The average produced ethanol concentration may vary depending on the ethanol fermentation system, the volume of the fermentation tank in which the reaction occurs, the concentration of the reactants placed into the fermentation tank, and other variables specific to the fermentation reaction. For example, the average produced ethanol concentration may vary between two fermentation tanks in the same ethanol production system if the two fermentation tanks have differing variables (e.g., size of fermentation vessel, concentration of reactants added, temperature, pH, lactic acid concentration, acetic acid concentration, etc.).

For a fermentation operation using the compositions and methods described herein, the ethanol production time may be decreased relative to the ethanol production time for a baseline fermentation operation without the use of nisin. The ethanol production time may be decreased due to the nisin compositions described herein controlling and/or reducing microbial populations that would otherwise compete with the yeast for nutrients necessary for producing ethanol. Therefore, the average baseline ethanol concentration can be reached sooner (i.e., the ethanol production time is decreased) using nisin as compared to a baseline fermentation operation without nisin.

In some examples, the ethanol production time is decreased about 0.1% to about 50%, about 0.1% to about 45%, about 0.1% to about 40%, about 0.1% to about 35%, about 0.1% to about 33%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, about 0.1% to about 1%, or about 0.1% to about 0.5%.

In some examples, the ethanol production time is decreased about 10 minutes to about 30 hours, about 10 minutes to about 28 hours, about 10 minutes to about 25 hours, about 10 minutes to about 22 hours, about 10 minutes to about 20 hours, about 10 minutes to about 18 hours, about 10 minutes to about 15 hours, about 10 minutes to about 12 hours, about 10 minutes to about 10 hours, about 10 minutes to about 8 hours, about 10 minutes to about 5 hours, about 10 minutes to about 1 hours, about 10 minutes to about 30 minutes, about 30 minutes to about 30 hours, about 1 hour to about 30 hours, about 2 hours to about 30 hours, about 5 hours to about 30 hours, about 8 hours to about 30 hours, about 10 hours to about 30 hours, about 12 hours to about 30 hours, about 15 hours to about 30 hours, about 18 hours to about 30 hours, about 20 hours to about 30 hours, about 22 hours to about 30 hours, about 25 hours to about 30 hours, or about 28 hours to about 30 hours. In some examples, an amount the ethanol production time is decreased depends on an initial amount of microbial population overgrowth prior to adding the nisin.

Without wishing to be bound by theory, it is believed that when the compositions described herein are added to the ethanol production system, the compositions provide antimicrobial effects to the ethanol production system by acting bactericidally. This action may be slowing the proliferation of the bacteria and/or reducing the bacterial populations within the ethanol production system. Slowing the proliferation of the bacteria and/or reducing the bacterial populations results in less competition with the yeast for nutrients necessary for producing ethanol and thereby increases a rate of ethanol production and decreases the ethanol production time.

As the compositions are added to various points of the ethanol production system, the intermediate material moving through the ethanol production system may encounter composition addition points at multiple stages through the ethanol production process. For example, if the compositions described herein are added to the slurry tank, growth of bacterial populations in the slurry tank may be controlled due to the addition of the nisin. After which, the slurry may be moved to a strainer, a jet cooker, a liquefaction tank, and so forth. As the intermediate material moves through these additional stages, the intermediate material may encounter additional sources of microbial contamination that can lead to proliferation of microbial populations. Additional amounts of the compositions described herein may be added downstream in the ethanol production system to address the additional microbial growth as the intermediate product in the ethanol production process moves throughout the system.

It is contemplated that the compositions described herein may be added at any stage of the ethanol production process that may be susceptible to bacterial overgrowth. The compositions described herein may be added once or multiple times to the ethanol production system. The present disclosure also contemplates using the compositions described herein in both batch fermentation and continuous fermentation operations. In some examples, the compositions described herein are added to a single fermentation vessel. In some examples, the compositions described herein may be added to multiple fermentation vessels. In some examples, the compositions described herein are added once, multiple times, intermittently, or as needed to the ethanol production system. The addition frequency and quantity may be dependent at least in part upon preset time periods for addition of the composition, measurements of desired or undesired concentration of various components in the ethanol production system, or in response to contamination concerns.

When the compositions are added to the ethanol production system as described herein, the compositions may be circulated through the ethanol production system. For example, when sufficient quantities of the compositions described herein are added to one or more vessels or components of the ethanol production equipment in the system, the composition may continue to act on the material for extended periods of time as it is processed and moves through the ethanol production system.

In some examples, the compositions described herein may exhibit longer term impacts on ethanol production beyond the immediate application of the compositions to the ethanol production system. For example, the compositions herein may demonstrate a backset effect.

In some examples, an increase in final ethanol concentration over the course of a fermentation operation may be a function of how the composition is dosed in the system. For example, the composition may be added at multiple locations, such as in a dosing vessel which directly feeds into the yeast propagation tank. Additionally, the composition may be added to a dosing vessel, pumped into the ethanol production system, then sent to multiple fermentation tanks. When the composition is added from a dosing vessel and pumped into the ethanol production system, it travels through a header, which is a series of interconnected passageways for components to be delivered to multiple vessels within an ethanol production system, such as multiple fermentation tanks. Delivery of the composition through the header may effectively clean the header, reducing microbial populations in the header that may proliferate and contaminate downstream vessels, such as fermentation tanks. Without wishing to be bound by theory, it is believed that by reducing the amount of microbial populations present in the header that may then be transferred to the fermentation tanks, more sugar will be available for the yeast to consume in the downstream tanks, and thus, over time, the final ethanol concentration may increase.

The compositions described herein may be added to the ethanol production system in a variety of ways. In some examples, the compositions are added to the ethanol production system in a pre-measured amount. The pre-measured amount may be a solid, a powder, or a liquid. In some examples, the pre-measured amount may be in a dissolvable package wherein the dissolvable package may be added whole into the ethanol production system without measurement of the composition or removal of the package. The dissolvable package may dissolve in the ethanol production system and release the composition into the system. The compositions may also be added to the ethanol production system by measuring out the amount of composition depending on the size and features of the ethanol production system, measured microbial population, or desired frequency of dosage.

In some examples, the compositions described herein may be added directly to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof. In some examples, the composition is added to a dosing vessel before the composition is added to the system. In some examples, the composition is added to a dosing vessel, and then is added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, directly from the dosing vessel. In some examples, the composition is mixed with water in the dosing vessel before the composition is added to the other vessels within the system described herein.

In some examples, the compositions described herein are continually dosed in the ethanol production system. In such examples, a constant or variable amount of the composition is continuously added to one or more parts of the ethanol production system.

In one example method for controlling microbial population in an ethanol production system, nisin may be added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof. In the example method, the nisin may be the only antimicrobial agent that is added to the ethanol production system during the fermentation process.

In one example method for controlling microbial population in an ethanol production system, a composition may be added to at least one of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof. The composition may comprise nisin and at least one surfactant.

The present disclosure provides advantages over conventional solutions by providing compositions and methods for controlling the growth of microbial population without the use of antibiotics, or reduced use of antibiotics, while using components that are GRAS and can be incorporated into by-product streams (e.g., animal feed, oil). In addition to being recognized as GRAS, the compositions described herein comprising nisin are also feed additive safe. Nisin may also function as an animal feed preservative as it may exit the ethanol production system as a by-product in animal feed.

Because the components within an ethanol production system often need to be GRAS, the byproducts of the system are important. In a dry grind facility, ethanol, distiller's dried grains with solubles (DDGS), and corn oil are generally sold as production outputs. DDGS is produced by processing the solids removed from the fermentation vessel after the fermentation reaction has been complete. These solids are typically removed from the fermentation vessel, sent to the beerwell, and stored in a whole stillage tank. From there, excess liquid is decanted off, and the resulting material is sent a drum dryer. DDGS is typically sold as animal feed. Corn oil is also produced by processing the solids that come out of the fermentation vessel after the fermentation reaction has been complete. These solids are typically removed from the fermentation vessel, sent to the beerwell, and stored in the whole stillage tank. From there, excess liquid is decanted off. That liquid is both water and “thin stillage”. While the removed water is eventually sent into the backset, the thin stillage is sent to the evaporators, where corn oil is extracted and eventually sold as a byproduct. As a result, the three components that dry grind ethanol facilities typically sell are: ethanol, DDGS, and corn oil. Thus, by decreasing the reaction time required to complete a single fermentation, more fermentation reactions can happen annually. The more fermentation reactions that can occur, the more ethanol, DDGS, and corn oil that can be produced, and thus the more revenue a plant can make. Measurements of output of ethanol DDGS, and corn oil can be helpful for evaluating the efficiency of an ethanol production system.

The compositions described herein may reduce or eliminate the need for antibiotics in an ethanol production system. In some examples, an ethanol production system in accordance with the compositions and methods described herein is free of antibiotics. In some examples, an ethanol production system in accordance with the compositions and methods described herein uses a reduced concentration of antibiotics.

The compositions described herein also may reduce the total sugar concentration in a fermentation reaction. As described herein, “total sugar” refers to the sum of the sugars DP4+, DP3, maltose, and glucose at any specified time point. For example, the time point may be at drop (completion) or at any other point during the fermentation operation. In a fermentation operation, the quantity of total sugar is highest at the beginning of the fermentation operation when the yeast have not yet used any sugar to begin producing ethanol. As the fermentation operation continues, the total amount of sugar decreases as the yeast use the sugar to make ethanol. At completion, almost all sugar should be consumed by the yeast to have a high concentration of ethanol and to avoid sending an excessive quantity of total sugar to the evaporators, which then must be cleaned. For example, the total sugar concentration at the end of fermentation (at drop) may be lower in comparison to a fermentation reaction without the compositions described herein. It may be beneficial for the total sugar concentration at the end of fermentation to be decreased to avoid sending as much sugar to the evaporators. The present disclosure contemplates using the compositions described herein to reduce total sugars at the end of fermentation.

Also described herein are systems of monitoring microbial growth in an ethanol production system. In some examples, a monitoring system may comprise one or more checkpoints for measuring microbial population in an ethanol production system.

In an example monitoring system, the compositions described herein may be applied to a vessel in the ethanol production system. The composition may act on the material in the vessel (for example, the composition acts on the slurry in the slurry tank) to control the microbial population in the vessel. As the material moves from the vessel downstream in the ethanol production system, there may be one or more checkpoints in which the microbial population is evaluated to determine if more of the composition should be added to the system. The checkpoints may comprise an operator who manually samples and checks the microbial population or a device that takes a sample and measures microbial population after a period of time or after a triggering event.

In such example monitoring system, the microbial population of the ethanol production system may be measured at one or more checkpoints throughout the ethanol production system to provide real-time feedback of the microbial population in the ethanol production system. When the microbial population is measured at the one or more checkpoints, if the microbial population exceeds a threshold value, more of the composition may be added to the ethanol production system.

The threshold value may be determined by regulations setting maximum tolerated microbial populations, set by operators at a particular ethanol production facility, and/or set based upon how the ethanol produced by the ethanol production system will be used (i.e., ethanol for one use may have a larger acceptable microbial population concentration than ethanol for another use).

If the microbial population exceeds the threshold value at any of the one or more checkpoints, an alert may be sent to notify operators at the ethanol production facility of the need to apply additional composition to the system. The alert may be delivered by a controller to a mobile device or other connected device. Such system provides for a real-time feedback loop that allows for as-needed dosing of the compositions described herein to provide continual microbial control.

The monitoring system may also be automated at the one or more checkpoints described herein. The one or more checkpoints may be automated in which machinery is installed that provides for automatic sampling at designated intervals to measure microbial population. The microbial measurement may be automated in which machinery is provided a program, reagents, and equipment to execute a microbial population measurement. The microbial population measurement may be compared to a predetermined threshold value of acceptable microbial population and a controller may evaluate if the measurement exceeds the threshold. If the measurement exceeds the threshold, the system may automatically send a signal that additional composition should be added to reduce the microbial population. The addition of the composition may also be automated, and a controller may determine an amount of the composition to add to the system based on a concentration-dependent relationship between the measured microbial population, the threshold value, the contents of the composition, and the system.

The present disclosure also contemplates use of nisin as part of a clean-in-place (CIP) system. CIP techniques are commonly used to clean ethanol production systems because they allow for the ethanol production equipment to be cleaned without disassembly of equipment, which otherwise leads to extended downtime that negatively impacts ethanol production.

Nisin and the other components described herein are contemplated as part of a CIP system. The components and compositions previously described herein are contemplated as part of a CIP system. The present disclosure also contemplates additional components that may be used for CIP cleaning.

In some examples, additional cleaning and sanitizing agents may be used in combination with nisin (and other components described herein) as part of a CIP cleaning system. CIP cleaning may be performed with one or more of peracetic acid, sodium hypochlorite, sodium hydroxide, potassium hydroxide, detergents, phosphoric acid, enzymes, hydrogen peroxide, quaternary ammonium compounds, alcohols, chelating agents, and other additives commonly used in CIP cleaning systems.

Nisin may be added to a CIP system in a variety of ways. Nisin may be added with other cleaning and sanitizing agents for a CIP system and circulated in the ethanol production system concurrently with the other components in the CIP system. Nisin may also be added separately as a separate step in a CIP system and allowed to circulate alone in the CIP system.

Nisin (alone or in combination with other CIP components) may circulate in the ethanol production system for about 1 minute to about 3 hours, about 1 minute to about 2 hours, about 1 minute to about 1 hour, about 1 minute to about 45 minutes, about 1 minute to about 30 minutes, about 1 minute to about 15 minutes, about 1 minute to about 5 minutes, about 5 minutes to about 3 hours, about 15 minutes to about 3 hours, about 30 minutes to about 3 hours, about 45 minutes to about 3 hours, about 1 hour to about 3 hours, or about 2 hours to about 3 hours.

Nisin may also remain in the ethanol production system after circulating. In some examples, after one or more steps of circulating nisin in the CIP system, the nisin may remain in the ethanol production system for a period of time until the equipment in the CIP system is used for another CIP cycle. In some examples, this period of time may be about 5 minutes to about 24 hours, about 5 minutes to about 16 hours, about 5 minutes to about 12 hours, about 5 minutes to about 10 hours, about 5 minutes to about 8 hours, about 5 minutes to about 6 hours, about 5 minutes to about 4 hours, about 5 minutes to about 2 hours, about 5 minutes to about 1 hour, about 5 minutes to about 30 minutes, about 30 minutes to about 24 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, or about 16 hours to about 24 hours.

In some examples, after nisin is used in the CIP system, the nisin may remain in the ethanol production equipment. For example, mash may be added on top of the nisin in the mash tank, coolers, or propagation tanks such that the nisin continues to act as a bacteriostat in the mash during fermentation.

The following non-limiting Examples are provided as illustrative embodiments of the disclosure. Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements. The Examples are illustrative and may only show limited numerical quantities of the components described above. Only some Examples are shown for the sake of brevity, but the full quantity ranges of components described above are contemplated.

### EXAMPLES

### Example 1: Measuring Efficacy of Nisin at Reducing Microbial Populations

In this Example, the efficacy of nisin was compared at various concentrations for control of the bacteria E. coli and S. aureus.

A minimally modified AOAC 960.09 standardized test method was used. AOAC 960.09 is a method prescribed by the Environmental Protection Agency (EPA) for evaluating the germicidal and detergent sanitation action of disinfectants, the entirety of which is incorporated herein.

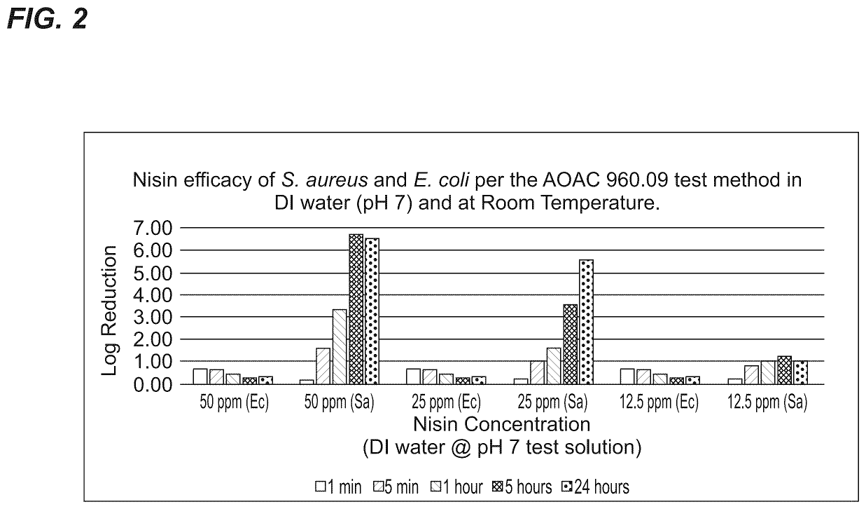

The standard AOAC 960.09 test method was modified to accommodate alternate test organisms, where applicable, and to allow for various exposure times or temperatures. One mL of prepared test culture was added to 99 mL of nisin testing solution and individual samples were taken and prepared in the concentrations shown and described below with respect to FIGS. 2-5 Solutions were prepared in sterile deionized water at various concentrations and pH levels to assess product effectiveness. Variable exposure temperatures and treatment times were analyzed as described below. All efficacy testing utilizing an adaptation of AOAC 960.09 was performed with the proper microbiological efficacy test controls to ensure sufficient test product neutralization and non-treated organism control count recovery.

FIGS. 2-5 show the efficacy of nisin at various concentrations and temperatures. In FIGS. 2-5, the concentrations of nisin are measured relative to the volume of the material in the fermentation vessel used in the test. The concentrations of nisin shown in FIGS. 2-5 also represent the actual concentration of the nisin, not the concentration of a nisin salt blend (for example, a concentration of 25 ppm nisin represents the concentration of nisin, and that nisin may have been part of a nisin salt blend that also comprised 75 ppm of a salt, such as sodium chloride). The concentrations of nisin tested are shown in FIGS. 2-5, and the activity of the nisin was about 1,000 IU/mg.

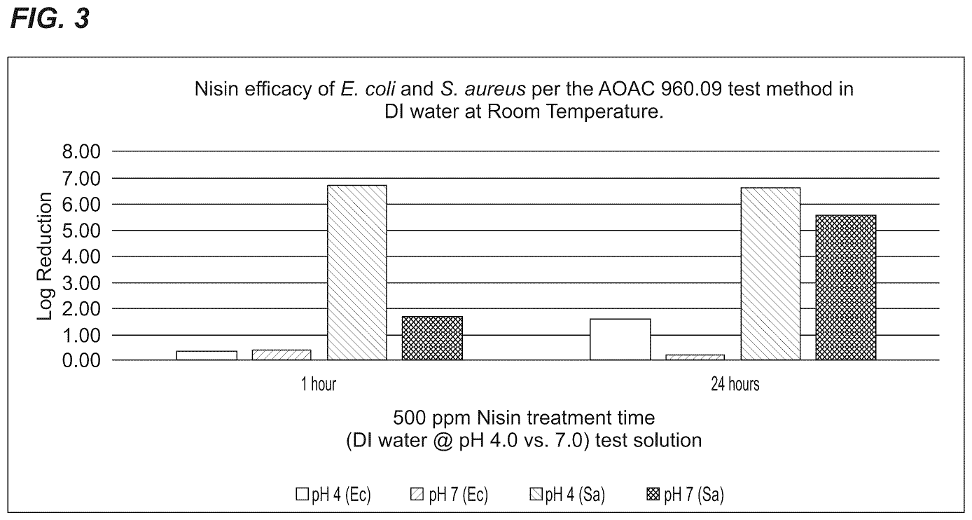

FIG. 2 shows the results of the above-described method where various concentrations of nisin were tested for efficacy over a periods of 24 hours against E. coli (Ec in FIGS. 2-3) and S. aureus (Sa in FIGS. 2-3) bacteria. The test was performed using deionized (DI) water, a pH of 7, and at room temperature.

At concentrations of 50 ppm of nisin, the composition demonstrated less than 1 log reduction of E. coli for all measured timepoints. E. coli is a gram negative bacterium, and nisin demonstrates excellent microbial control for gram positive bacteria. Therefore, it is unsurprising that nisin provided limited (though a non-zero amount of) microbial population control of E. coli.

However, at concentrations of 50 ppm of nisin, the composition demonstrated significant log reduction of S. aureus, a gram positive bacterium. The composition provided some reduction in microbial population after 1 minute, over a 1 log reduction after 5 minutes, over a 3 log reduction after 1 hour, and over a 6 log reduction after 5 hours and 24 hours. This demonstrates that at concentrations of 50 ppm, nisin provides high levels of microbial population control of gram positive bacteria after as little as 5 minutes.

At concentrations of 25 ppm and 12.5 ppm, nisin produced limited, but not zero, log reduction in microbial populations of E. coli, a gram negative bacterium. However, even at lower concentrations of 25 ppm and 12.5 ppm, nisin demonstrated effective log reduction of S. aureus. The results of this test demonstrate that nisin provides effective microbial control of gram positive bacteria at various concentration ranges and time periods.

FIG. 3 shows the efficacy of nisin at various pH levels for E. coli and S. aureus. For E. coli samples, nisin demonstrated limited microbial reduction after 1 hour at both pH 4 and pH 7, though after 24 hours, nisin demonstrated nearly a 2 log reduction in E. coli at a pH of 4. While nisin is most effective against gram positive bacteria, the results of this test show that nisin demonstrates some efficacy against gram negative bacteria.

For the S. aureus samples, nisin demonstrated over a 6 log reduction after 1 hour at a pH of 4, and over a 1 log reduction after 1 hour at a pH of 7. After 24 hours, nisin demonstrated over a 6 log reduction at a pH of 4 and over a 5 log reduction at a pH of 7. The results of this test demonstrate that nisin maintains its efficacy over a period of 24 hours and across multiple pH levels, which is advantageous for use in an ethanol production system in which the pH may vary extensively, and the ethanol production process may be lengthy.

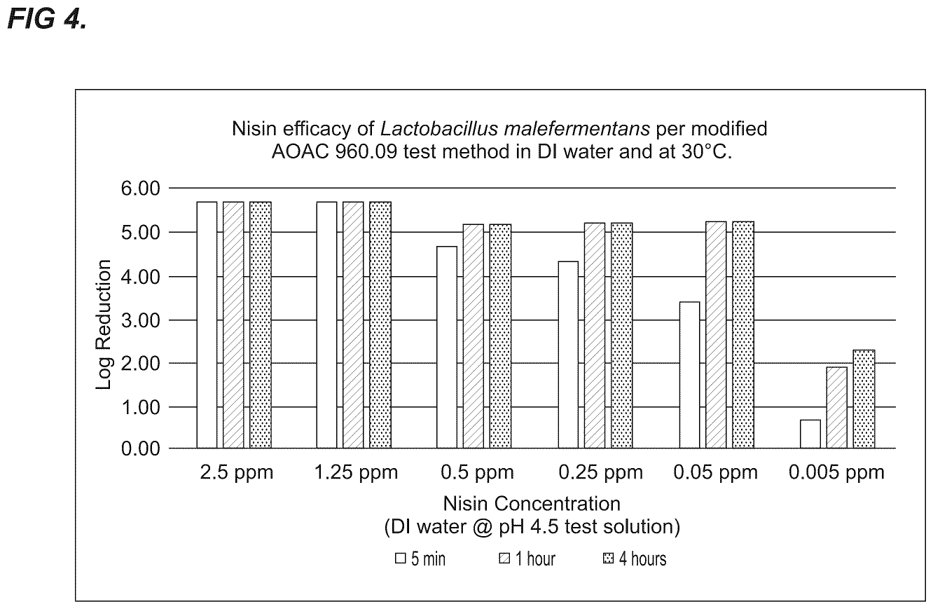

The efficacy of nisin against Lactobacillus malefermentans (L. malefermentans) was also measured at elevated temperatures of 30° C. in DI water at a pH of 4.5. The results of this test are shown in FIG. 4, in which concentrations of nisin from 0.005 ppm to 2.5 ppm were tested after 5 minutes, 1 hour, and 4 hours.

At concentrations of 2.5 ppm and 1.25 ppm, the nisin demonstrated over a 5 log reduction in L. malefermentans after 5 minutes, 1 hour, and 4 hours. At concentrations of 0.5 ppm, 0.25 ppm, and 0.05 ppm the nisin demonstrated over a 5 log reduction in L. malefermentans after 1 hour and 4 hours, and over 3 log reduction after 5 minutes. When the concentration of nisin was lowered to 0.005 ppm, the nisin still demonstrated a reduction in L. malefermentans at all time points, though the log reduction was noticeably less at this lower concentration. The results of this test demonstrate that nisin is effective at controlling L. malefermentans populations, even at low concentrations and after short time periods.

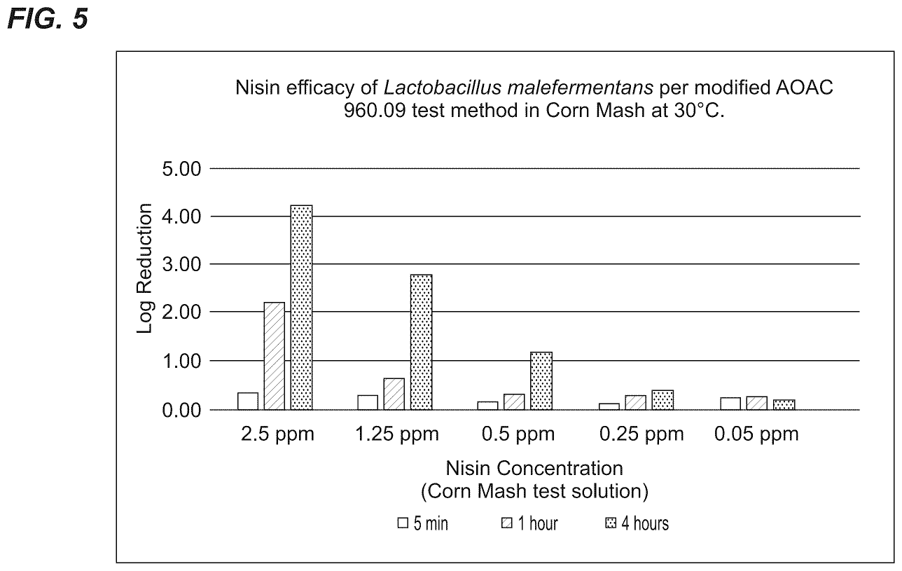

The efficacy of nisin was also tested against L. malefermentans in corn mash at 30° C., the results of which are shown in FIG. 5. One mL of Lactobacillus malefermentans culture was added to 98 mL of sterilized corn mash sample. To simulate the addition of nisin to a mash tank, nisin was added in concentrations of 0.05 ppm to 2.5 ppm to corn mash and its efficacy was measured.

At concentrations of nisin 0.25 ppm to 2.5 ppm, the efficacy of the nisin increased as the nisin remained in the corn mash for longer periods of time, with the nisin demonstrating highest efficacy after 4 hours. The nisin was most effective at reducing the population of L. malefermentans in concentrations of 2.5 ppm after 4 hours, with a log reduction of over 4, however, nisin still provided some efficacy at low concentrations of 0.05 ppm and 0.25 ppm.

The results of FIGS. 2-5 suggest that nisin provides microbial population control across varying concentration ranges, temperatures, pHs, and in different environments (i.e., with DI water and in corn mash). While only concentrations of 0.005 ppm to 50 ppm were tested in these examples discussing bench testing, it is expected that higher concentrations of nisin will impart similar effects on a larger scale.

### Example 2: Evaluation of Nisin for Yeast Compatibility

As discussed herein, the compositions comprising nisin are yeast safe or yeast compatible, meaning that the compositions do not significantly damage the yeast necessary for fermentation.

Various concentrations of nisin were tested in accordance with the minimally modified AOAC 960.09 method described in Example 1. This testing was conducted over a period of 5 minutes to 4 hours to evaluate the effect of nisin on Saccharomyces cerevisiae yeast.

TABLE 1

| Nisin | Use solution | | | | Log |
|---|---|---|---|---|---|
| concentration | pH | Temperature | Time | | reduction |
| 25 ppm | 4.5 | 30 C. | 5 | minutes | 0.00 |
| | | | 1 | hour | 0.02 |
| | | | 4 | hours | 0.00 |
| 10 ppm | | | 5 | minutes | 0.00 |
| | | | 1 | hour | 0.18 |
| | | | 4 | hours | 0.07 |
| 1 ppm | | | 5 | minutes | 0.03 |
| | | | 1 | hour | 0.01 |
| | | | 4 | hours | 0.07 |

As shown in Table 1, at concentrations of nisin from 1 ppm to 25 ppm, there is minimal log reduction of the yeast. This demonstrates that nisin is yeast safe or yeast compatible and can be used in an ethanol production system with minimal damage to the yeast needed for fermentation, while still providing benefits (as shown in Example 1).

### Example 3: Evaluation of Nisin Effects on Ethanol, Lactic Acid, and Acetic Acid Content

In this Example, compositions prepared in accordance with the present disclosure was added to a small scale fermentation batch to evaluate efficacy of controlling microbial populations.

About 400 grams of corn were ground and added to about 2900 to 3000 mL of water that was heated to greater than 74° C. in a one gallon glass jug. The ground corn and water mixture was held at 74° C. for 5 minutes using a water bath, after which the temperature was allowed to drop below 67° C. using a cold water bath. 100 grams of malted barley and 20 grams of yeast were added to the mixture and stirred. The fermentation mixture was then cooled to below 30° C. using a cold water bath. 20 grams of yeast was added to the mixture, stirred for 2 minutes, and a one-way airlock was placed on the glass jug such that carbon dioxide could escape the jug but outside air could not enter. The glass jug was placed in a 35° C. oven. This fermentation mixture was divided into several groups to evaluate microbial control abilities of various compositions.

A test was conducted to compare the performance of a control sample, a sample treated with 1.05 ppm nisin with an activity of about 1,000 IU/mg, and a sample treated with 10.5 ppm nisin with an activity of about 1,000 IU/mg over the course of 48 hours. The ethanol content, lactic acid content, and acetic acid content were measured for each sample over the course of 48 hours.

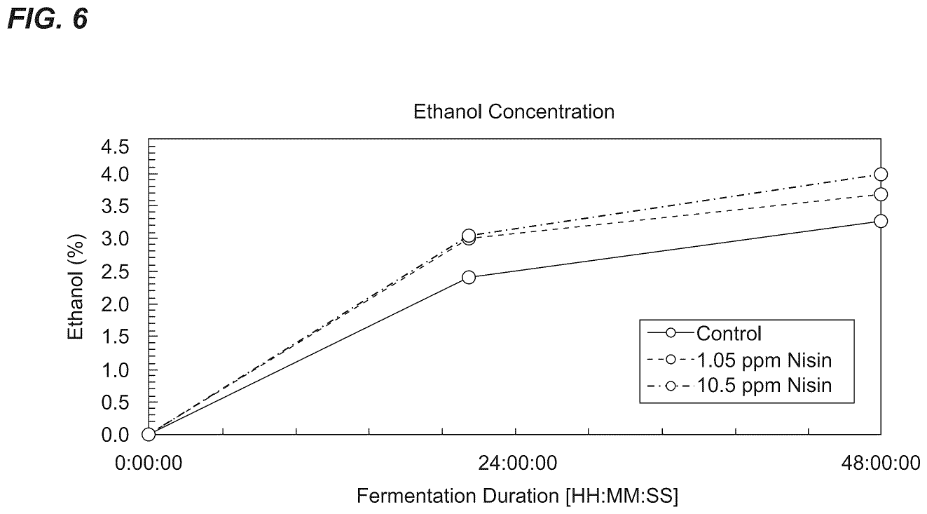

FIG. 6 shows the percent ethanol content in the fermentation mixture for each sample over the course of 48 hours. The control sample produced the lowest ethanol concentration over the course of the 48 hours, while the samples treated with 1.05 ppm and 10.5 ppm nisin demonstrated similar, higher concentrations of ethanol over the course of 48 hours.

This suggests that the addition of nisin at various concentrations results in a higher ethanol content as compared to a control without nisin treatment. It is believed that this is due to nisin controlling and/or reducing microbial population because the microbial population would otherwise compete with the yeast for nutrients necessary for producing ethanol.

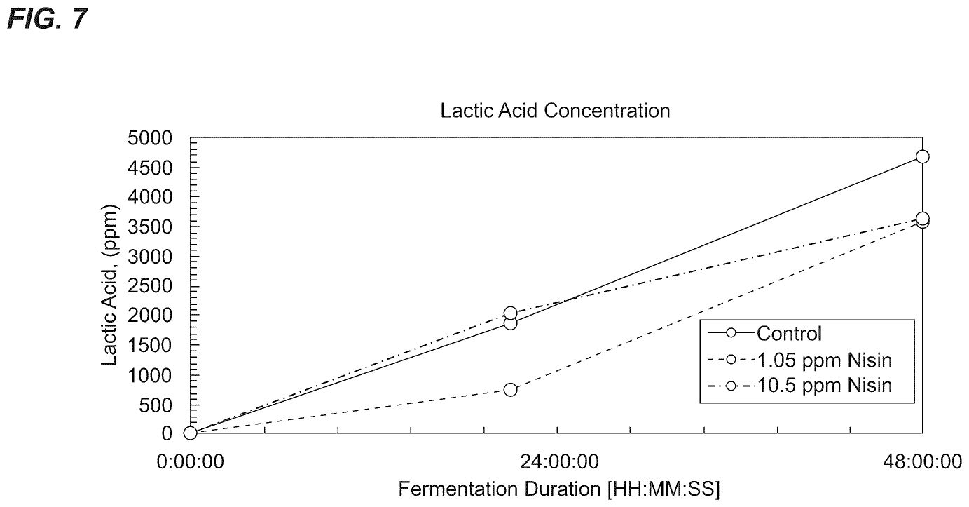

FIG. 7 shows the lactic acid content in the fermentation mixture for each sample. Lactic acid is produced by lactic acid-producing bacteria, such as bacteria from the genus Lactobacillus, and is generally undesirable in fermentation as the lactic acid-generating bacteria compete with the yeast for nutrients and produce the unwanted byproduct lactic acid.

The control sample demonstrated a large increase in lactic acid production over the course of 48 hours. The sample treated with 10.5 ppm of nisin demonstrated a notable decrease in lactic acid production as compared to the control sample after 48 hours. The sample treated with 1.05 ppm also demonstrated a decrease in lactic acid as compared to the control sample, with comparable lactic acid reduction after 48 hours. This suggests that concentrations as low as 1.05 ppm can provide beneficial lactic acid reduction.

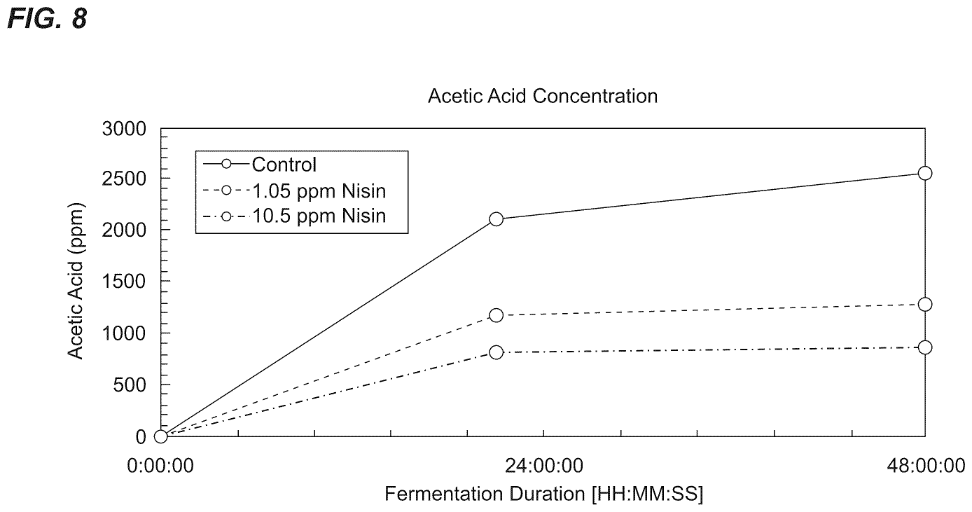

FIG. 8 shows the acetic acid content in the fermentation mixture for each sample. Acetic acid is produced by acetic acid-producing bacteria, such as Acetobacter aceti, and is also generally undesirable in fermentation as the bacteria can compete with yeast for nutrients and produce unwanted acetic acid.

In FIG. 8, the control sample demonstrated a large increase in acetic acid production over the course of 48 hours. The sample treated with 10.5 ppm nisin demonstrated the largest reduction in acetic acid content, but the sample treated with 1.05 ppm nisin also demonstrated considerable reduction in acetic acid content as compared to the control sample.

The results of this test suggest that nisin can provide reduction in microbial populations that produce acetic acid. However, as described herein, nisin is particularly beneficial at targeting gram positive bacteria, such as Lactobacillus. Nisin appears to still demonstrate some efficacy against gram negative bacteria, such as acetic acid-producing bacteria, as shown in FIG. 8 and in Example 1.

### Example 4: Comparison of nisin with other antimicrobial agents for ethanol, lactic acid, and acetic acid content

In this Example, compositions prepared in accordance with the present disclosure were added to an alternative small scale fermentation batch to evaluate efficacy of controlling microbial populations. The alternative small scale fermentation batch was prepared differently than the batch in Example 3 to modify test conditions to better simulate conditions in a full-scale ethanol production system.

About 150 grams of corn were ground and added to about 700 mL of water that was heated to greater than 74° C. in a one liter plastic bottle. The mixture of the corn and water was shaken in the bottle to produce a suspension between the corn and water. The bottle was placed in a hot water bath at 76° C. for 10 minutes with intermittent shaking to maintain the suspension. The bottle was removed from the hot water bath and allowed to cool to 67° C. 50 grams of malted barley was added to the bottle and shaken to mix the barley with the suspension. The bottle was placed in an ice water bath to reduce the temperature of the mixture to below 30° C. 5 grams of yeast was added and shaken, and an airlock was placed on the bottle. Nine batches of this fermentation mixture were produced to test the various treatments discussed below, each of which were tested in triplicates and averaged for data collection.

A test was conducted to compare the performance of a control sample, a sample treated with 0.63 ppm nisin at about 1,000 IU/mg, a sample treated with 15 ppm chlorine dioxide (ClO₂), and a sample treated with 0.01% to 1.0% hop acid. The ethanol content, lactic acid content, and acetic acid content were measured for each sample over the course of 48 hours.

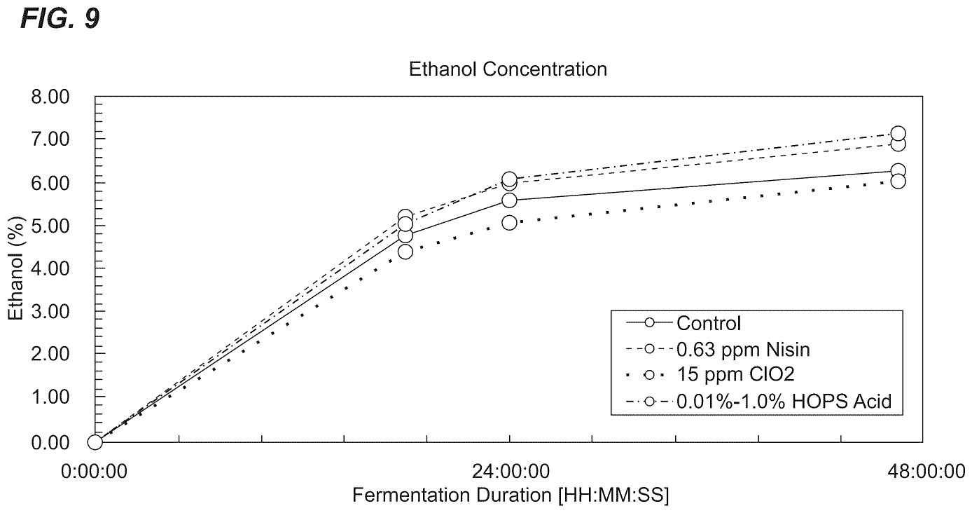

FIG. 9 shows the ethanol produced for each of the four treatments over the course of 48 hours. The sample treated with 15 ppm of ClO₂ demonstrated the lowest ethanol content after 48 hours, even lower than the ethanol content of the control sample, which suggests that ClO₂ is not an effective antimicrobial for improving ethanol production content in an ethanol production system. This also suggests that ClO₂ may be killing the yeast, which also lowers ethanol production relative to the control sample.

The samples treated with 0.63 ppm nisin and 0.1% to 1.0% hop acid demonstrated similar increases in ethanol production greater than the ethanol produced by the control sample, which suggests that both the nisin and hop acid were effective in increasing ethanol content by controlling microbial populations that interfere with ethanol production.

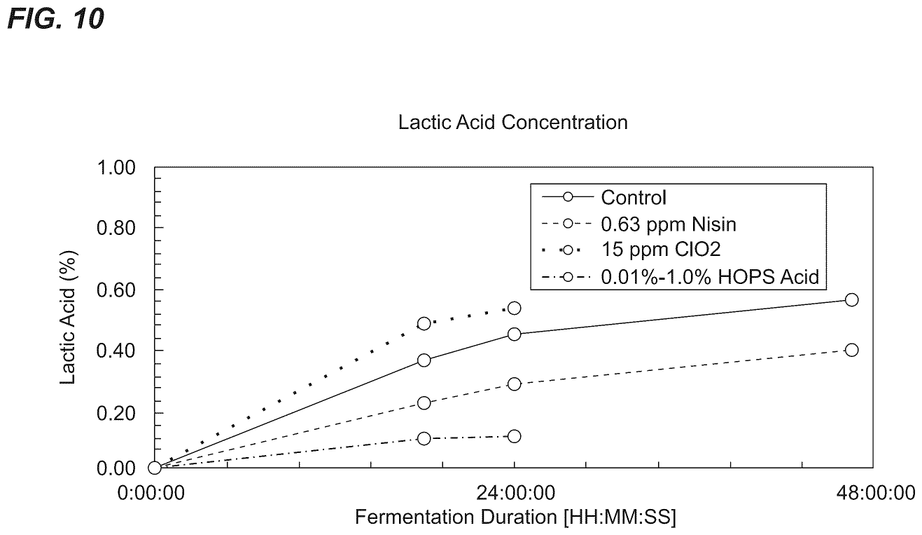

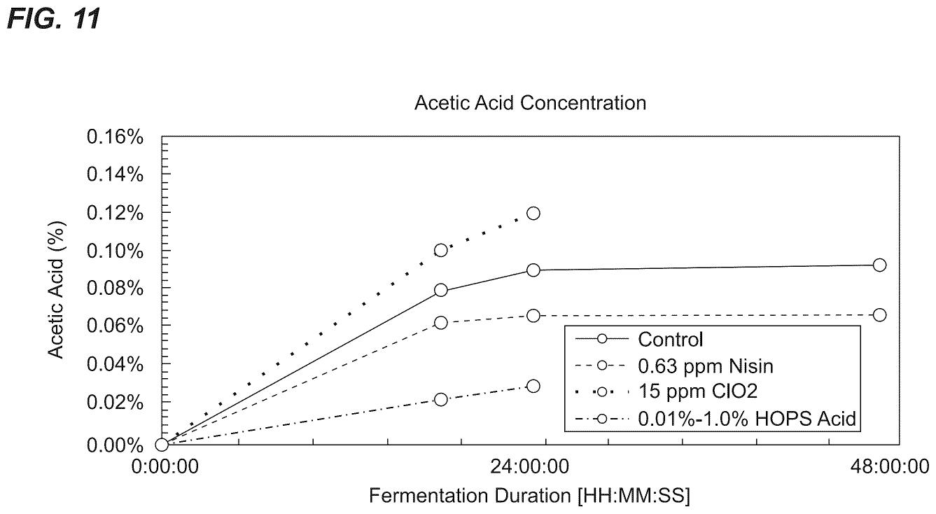

FIGS. 10 and 11 show the lactic acid content and the acetic acid in each of the four samples over the course of 48 hours, though lactic acid and acetic acid content for ClO₂ and hop acid were only measured through 24 hours. The results of FIGS. 10 and 11 show that ClO₂ is not an effective antimicrobial agent for reducing lactic acid content. Both nisin and hop acid demonstrated a decrease in lactic acid and acetic acid as compared to the control sample, with hop acid demonstrating superior reduction in lactic acid and acetic acid.

The results of this test suggest that nisin and hop acid provide beneficial decreases in lactic acid and acetic acid content as compared to a control sample.

### Example 5: Comparison of nisin with other antimicrobial agents for ethanol, lactic acid, and acetic acid content

A fermentation mixture was prepared as described in Example 4 and divided into eight groups. A test was conducted to compare the performance of a control sample, a sample treated with 1.20 ppm antibiotics, 0.53 ppm nisin at about 1,000 IU/mg, 1.05 ppm nisin at about 1,000 IU/mg, 2.10 ppm nisin, 12.5 ppm ClO₂, 25 ppm ClO₂, and 50 ppm ClO₂. The ethanol content, lactic acid content, and acetic acid content were measured for each sample over 48 hours.

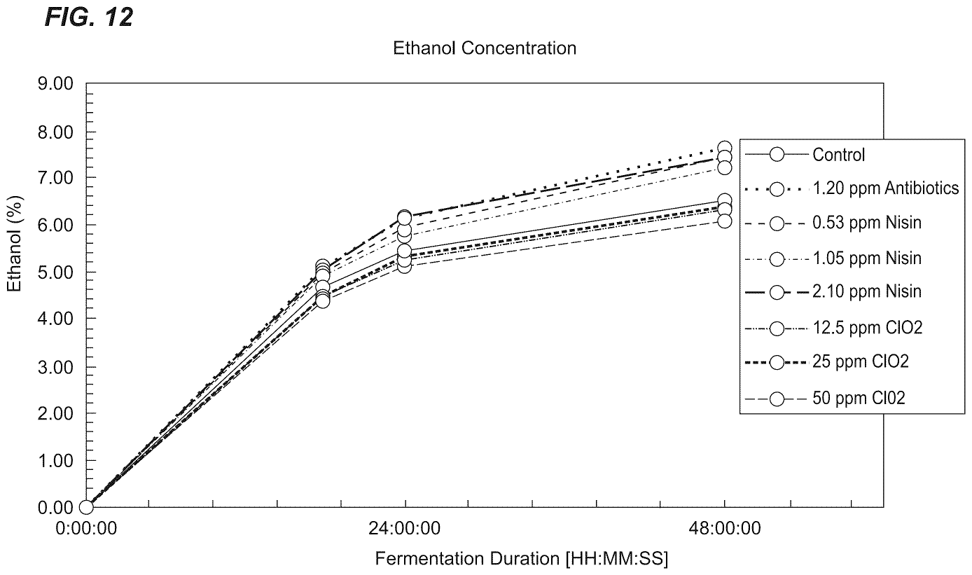

FIG. 12 shows the ethanol content for each sample over the course of 48 hours. The samples treated with ClO₂ demonstrated the lowest ethanol content compared to the other samples, which suggests that ClO₂ is not an effective antimicrobial for reducing microbial populations in ethanol fermentation.

The samples treated with antibiotics and all concentrations of nisin demonstrated similar ethanol contents that were greater than the ethanol content for the control sample, which suggests that the antibiotics and nisin both provide microbial population reduction that improved ethanol content as compared to the control sample. This also suggests that nisin provides similar microbial population reduction as compared to antibiotics, which is beneficial for reducing antibiotic use while reducing microbial populations.

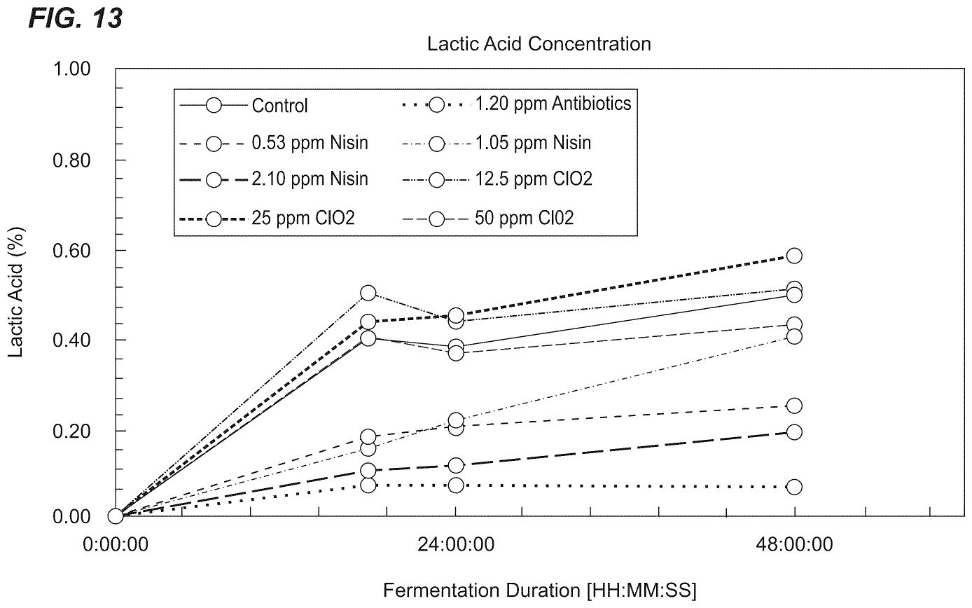

FIG. 13 demonstrates the lactic acid content for each of the samples over the course of 48 hours. The samples treated with ClO₂ and the control sample demonstrated the highest lactic acid content over the course of 48 hours, which further suggests that ClO₂ is not an effective antimicrobial agent for an ethanol production system. The sample treated with antibiotics demonstrated the lowest lactic acid content and thus the best microbial population reduction of lactic acid-producing bacteria, however, the sample treated with 2.10 ppm nisin demonstrated notable lactic acid content reduction as well. This suggests that while nisin (at the tested concentrations) may not be as effective at reducing lactic acid-producing bacteria as antibiotics, nisin provides an alternative solution for antibiotics to significantly reduce lactic acid-producing bacteria.

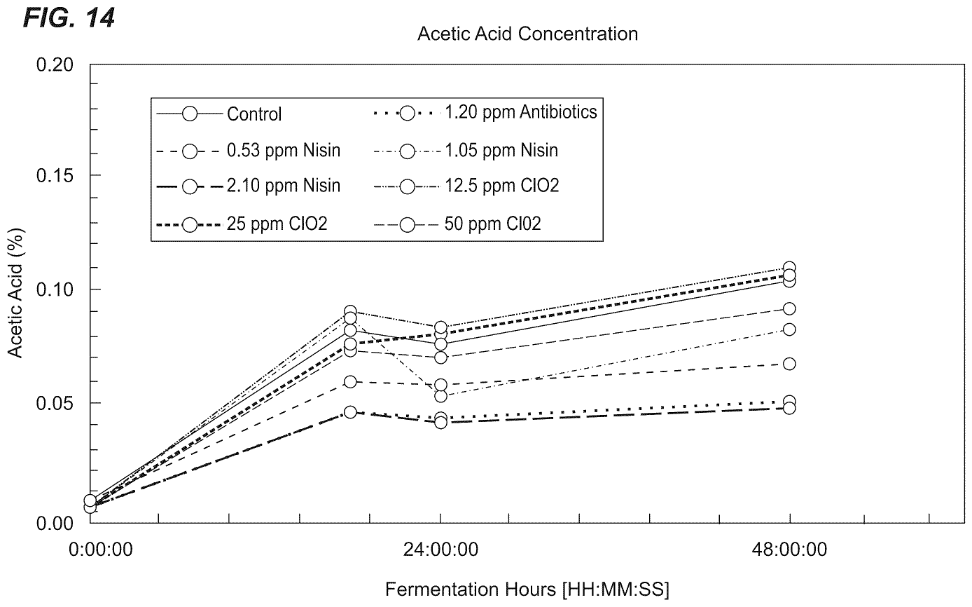

FIG. 14 demonstrates the acetic acid content for each of the samples over the course of 48 hours. The samples treated with ClO₂ and the control sample demonstrated the highest acetic acid content over the course of 48 hours, which further suggests that ClO₂ is not an effective antimicrobial agent for an ethanol production system. The samples treated with nisin demonstrated acetic acid reduction compared to the control sample, while the 2.10 ppm nisin sample provided near identical acetic acid reduction as compared to the sample treated with antibiotics. This suggests that nisin may provide comparable acetic acid reduction as compared to antibiotics, which provides an alternative to using antibiotics.

### Example 6: Evaluation of Nisin Effects on the Rate of Ethanol Production: Site 1

In this Example, compositions prepared in accordance with the present disclosure were added to a large scale fermentation system to evaluate the effects of nisin on ethanol production rate at a first ethanol production facility, Site 1.

A test was conducted over the course of 66 hours to compare the performance of large scale baseline fermentation without the addition of nisin with a large scale fermentation trial using nisin. The ethanol and sugar concentrations were measured over the course of 66 hours.

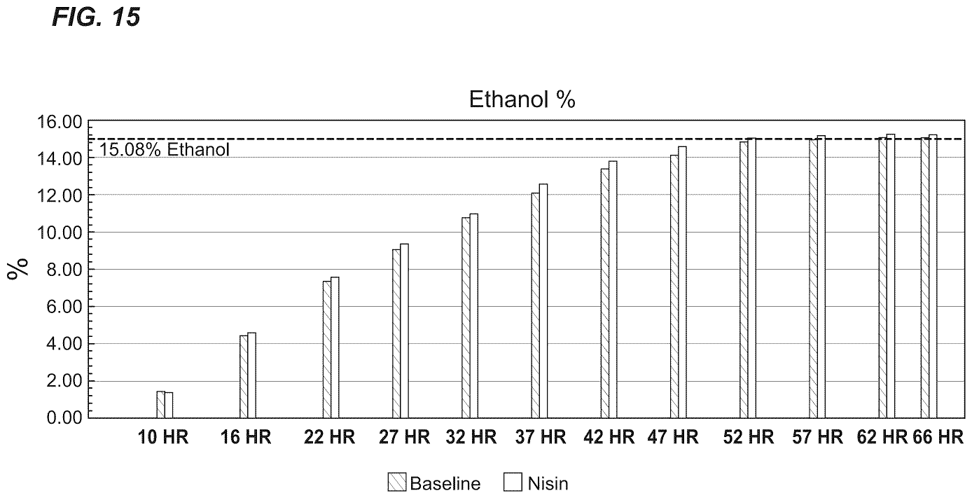

Baseline fermentation was determined over a period of time, in which the normal fermentation operations of Site 1 were performed. Data was collected for each operation over the course of the normal fermentation time, such as data regarding ethanol concentration and total sugar concentration. The baseline fermentation at Site 1 included antibiotics. This data is shown in FIGS. 15-18 and is discussed further below.

A fermentation operation with nisin was then performed. About 30 pounds (lbs) of a nisin composition was added to the fermentation system: one lb was added to the yeast propagation tank and 29 lbs were added to a dosing vessel, which fed directly into the fermentation tank. In total, 0.290 ppm of nisin were added with an activity of about 1,000 IU/mg. The fermentation tank had a volume of 730,000 gallons (gal) and was estimated to be filled to 95% capacity for fermentation (about 693,500 gal).

The ethanol concentration and total sugar concentration were measured over the course of 66 hours after nisin was initially added to the ethanol fermentation system. The results are shown in FIGS. 15-18.

The following baseline data points shown in FIGS. 15-18 were determined through linear interpolation: 16-HR, 27-HR, 37-HR, 47-HR, 57-HR, as defined in Eqn. 1:

$${Y}_{t} = {Y}_{B} + \frac{{Y}_{A} - {Y}_{B}}{{X}_{A} - {X}_{B}} * ( {X}_{t} - {X}_{B} ) \qquad ( 1 )$$

where Yₜ is the metric of interest, Y_{A} is the metric at the closest measured timepoint above time, t, Y_{B} is the metric at the closest measured timepoint below time, t, X_{A} is the fermentation time of closest to the measured timepoint above time, t, X_{B} is the fermentation time of the closest measured timepoint below time, t, and Xₜ is the fermentation time of interest that was not measured.

FIG. 15 shows, over the course of 66 hours, the ethanol concentration in the baseline fermentation and the fermentation trial with the added nisin. The dotted line extending across the plot at 15.08% ethanol represents the average baseline final ethanol concentration for Site 1, in this fermentation vessel. The fermentation trial with the added nisin produced a higher concentration of ethanol at all time points after 10-HR. This suggests that adding nisin may result in a higher ethanol concentration as compared to a baseline without nisin treatment, in about the same amount of time, in some fermentation reactions. Thus, this suggests that adding nisin increases the rate of ethanol production.

Without wishing to be bound by theory, it is believed that this is due to nisin controlling and/or reducing microbial population because the microbial population would otherwise compete with the yeast for nutrients necessary for producing ethanol.

In some fermentations at Site 1, the addition of nisin did not produce a notable increase in ethanol concentration as compared to the baseline without nisin treatment. Without wishing to be bound by theory, this may be due to a variety of factors, including but not limited to, cleanliness of the equipment, percent of corn (or other high carbohydrate source) solids, temperature, pH, etc.

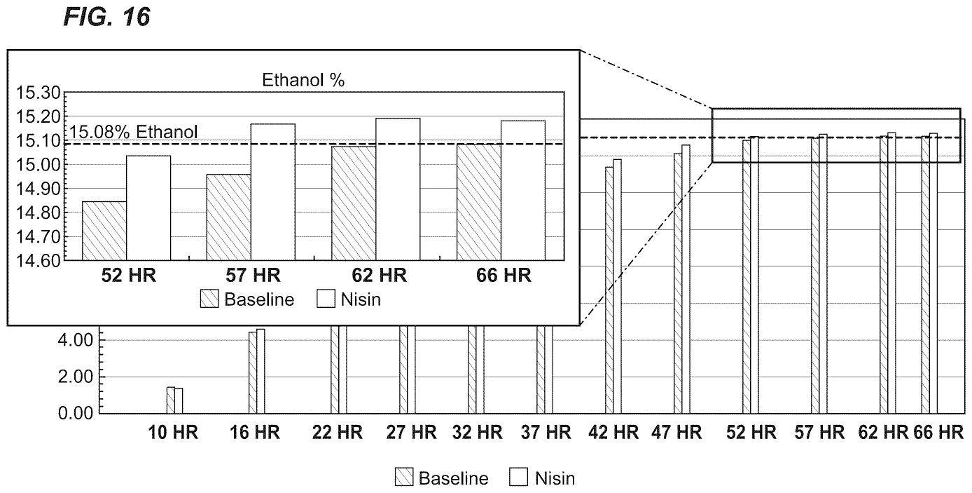

FIG. 16 is an expanded view of FIG. 15 and shows that the fermentation trial with the added nisin, at completion (66-HR), produced a higher concentration of ethanol than the average baseline ethanol concentration at completion, which is 15.08%. This also suggests that nisin can be added to the fermentation process to produce substantially the same quantity of ethanol (e.g., the average baseline ethanol concentration) at a faster rate.

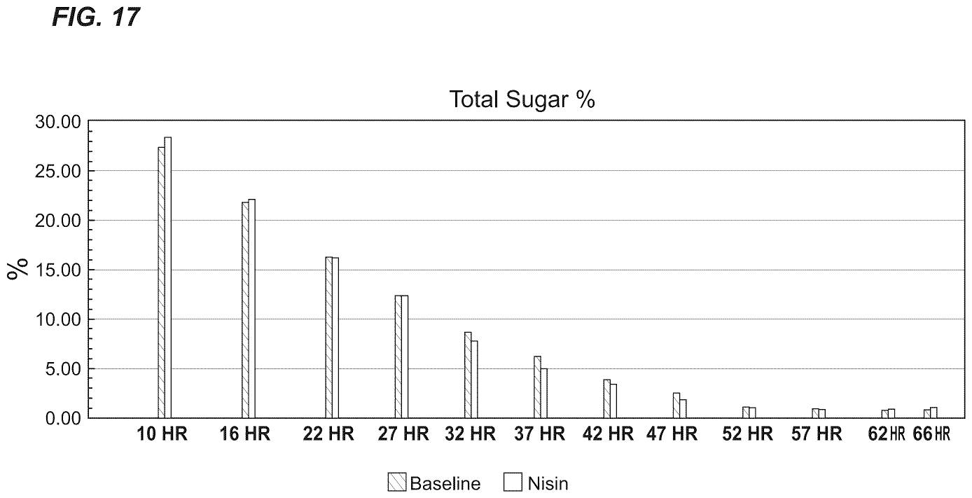

FIG. 17 shows, over the course of 66 hours, the total sugar concentration in the baseline fermentation and the fermentation trial with the added nisin.

Both the baseline fermentation trial and the fermentation trial with the added nisin began with about the same amount of total sugar. As shown in FIG. 17, over the course of 66 hours, the total sugar concentration decreases in both the baseline fermentation and the fermentation trial with the added nisin.

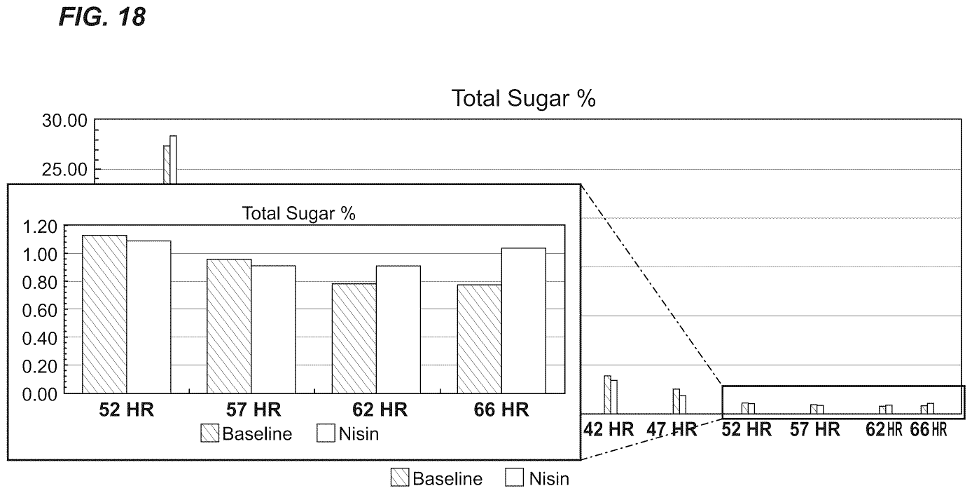

FIG. 18 is an expanded view of FIG. 17 and shows that the fermentation trial with the added nisin has about the same total sugar concentration at completion as the baseline fermentation. This suggests that the nisin did not meaningfully impact the final total sugar concentration at the end of the fermentation process in this particular trial, but rather how quickly the process occurs. It is expected that some trials will result in a reduction in total sugars at the end of fermentation.

### Example 7: Evaluation of Nisin Effects on the Rate of Ethanol Production: Site 2

In this Example, compositions prepared in accordance with the present disclosure were added to a large scale fermentation system to evaluate the effects of nisin on ethanol production rate at a second ethanol production facility, Site 2. The test site and conditions were different from those of Example 6.

A test was conducted, over the course of 83 hours, to compare the performance of large scale baseline fermentation without the addition of nisin with a large scale fermentation trial using nisin. The ethanol and sugar concentrations were measured over the course of 83 hours.

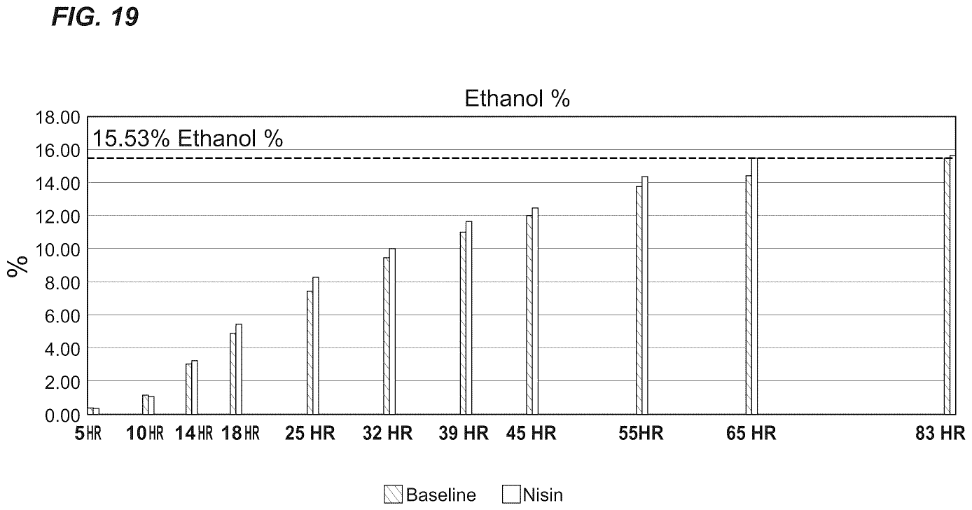

Baseline fermentation was determined over a period of time, in which the normal fermentation operations of Site 2 were performed. Data was collected for each operation over the course of the normal fermentation time, for example, regarding ethanol concentration and total sugar concentration. The baseline fermentation at Site 2 included antibiotics. This data is shown in FIGS. 19-22 and is discussed further below.

A fermentation operation with nisin was then performed. About 45 lbs of a nisin composition was added to the fermentation system: one lb was added to the yeast propagation tank and 44 lbs were added to a dosing vessel which fed directly into the fermentation tank. The nisin composition comprised about 0.387 ppm with an activity of about 1,000 IU/mg. The fermentation tank had a volume of 806,000 gal and was estimated to be filled to 96.7% capacity for fermentation (about 780,000 gal).

The ethanol concentration and total sugar concentration were measured over the course of 83 hours after nisin was initially added to the ethanol fermentation system. The results are shown in FIGS. 19-22.

The following baseline data points shown in FIGS. 19-22 were determined through linear interpolation: 5-HR, 10-HR, 18-HR, 25-HR, 32-HR, 39-HR, 55-HR, 83 HR, as defined in Eqn. 1 of Example 6.

FIG. 19 shows, over the course of 83 hours, the ethanol concentration in the baseline fermentation and the fermentation trial with the added nisin. The dotted line extending across the plot at 15.53% ethanol represents the average baseline final ethanol concentration for Site 2, in this fermentation vessel. The fermentation trial with the added nisin produced a higher concentration of ethanol at all time points after 10-HR. This suggests that adding nisin may result in a higher ethanol concentration as compared to a baseline without nisin treatment, in about the same amount of time, in some fermentation reactions. Thus, this suggests that adding nisin increases the rate of ethanol production.

Without wishing to be bound by theory, it is believed that this is due to nisin controlling and/or reducing microbial population because the microbial population would otherwise compete with the yeast for nutrients necessary for producing ethanol.

In some fermentations, the addition of nisin may not produce a notable increase in ethanol concentration as compared to the baseline without nisin treatment. Without wishing to be bound by theory, this may be due to a variety of factors, including but not limited to, cleanliness of the equipment, percent of corn (or other high carbohydrate source) solids, temperature, pH, etc.

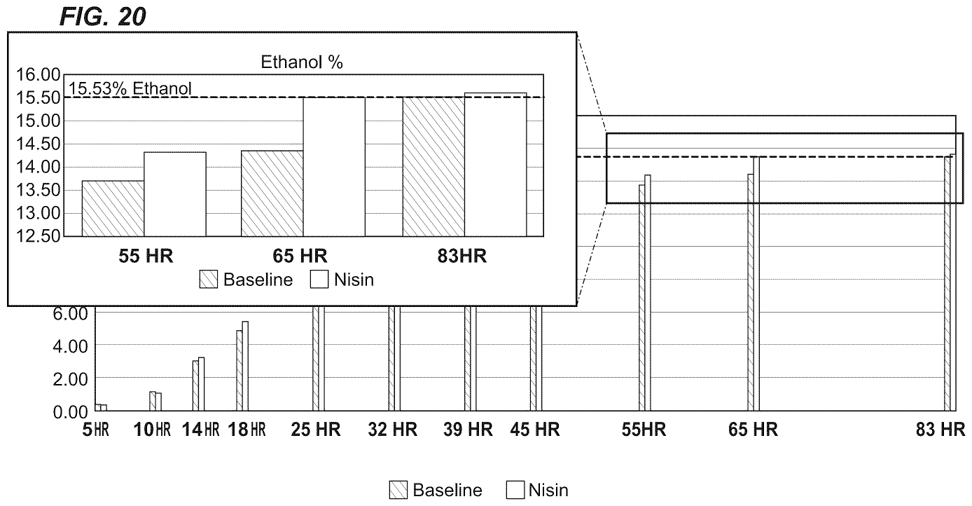

FIG. 20 is an expanded view of FIG. 19 and shows that the fermentation trial with the added nisin, at completion (83-HR), produced a higher concentration of ethanol than the average baseline ethanol concentration at completion, which is 15.53%. Moreover, the average baseline ethanol concentration of was essentially reached at the 65-HR timepoint for the nisin sample, as opposed to the baseline sample reaching the average baseline ethanol concentration at the 83-HR timepoint. This also suggests that nisin can be added to the fermentation process to produce the same quantity of ethanol at a faster rate.

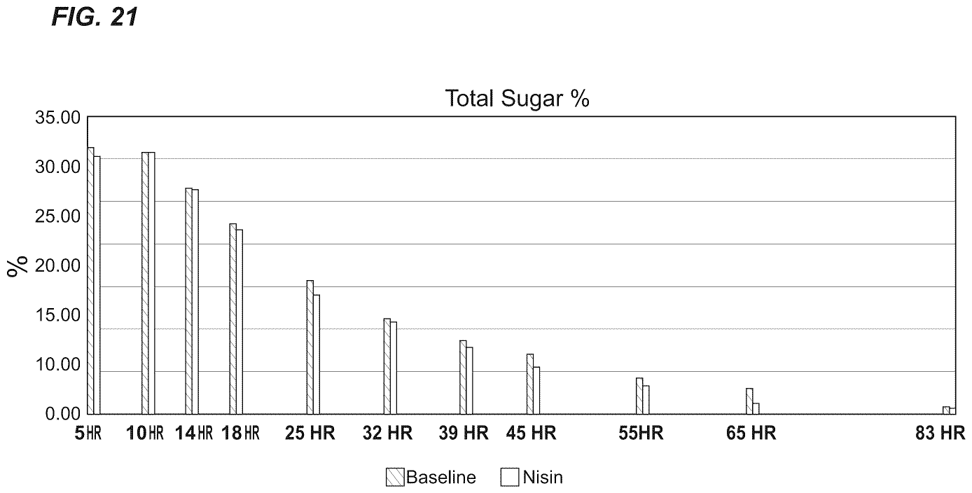

FIG. 21 shows, over the course of 83 hours, the total sugar concentration in the baseline fermentation and the fermentation trial with the added nisin. Both the baseline fermentation trial and the fermentation trial using nisin began with about the same quantity of total sugar. As shown in FIG. 21, over the course of 83 hours, the total sugar concentration decreases in both the baseline fermentation and the fermentation trial with the added nisin.

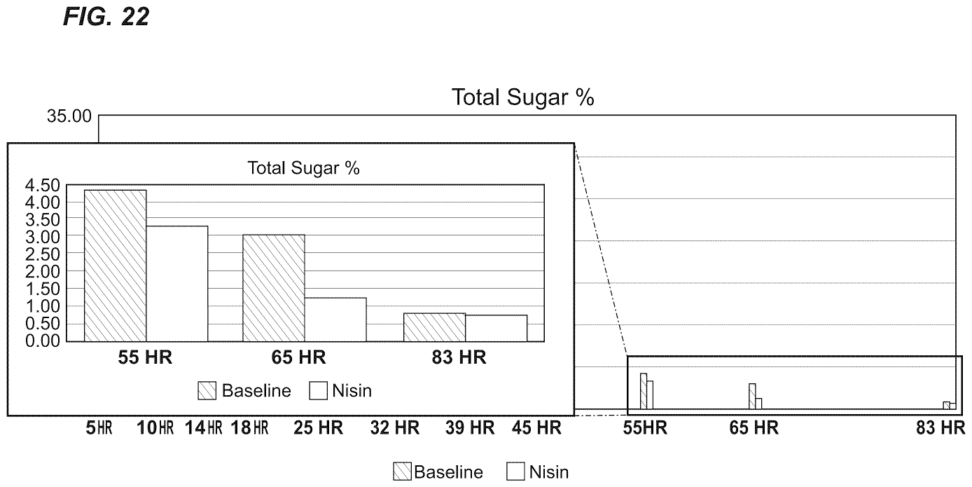

FIG. 22 is an expanded view of FIG. 21 and shows that the fermentation trial with the added nisin has about the same total sugar concentration at completion as the baseline fermentation. This suggests that the nisin does not meaningfully impact the final total sugar concentration at the end of the fermentation process, but rather how quickly the process occurs.

### Example 8: Evaluation of Nisin Effects on Ethanol Production: Site a and Site B

In this Example, compositions prepared in accordance with the present disclosure were added to a large scale fermentation system to compare the effects of nisin on ethanol production in two ethanol production facilities, Site A and Site B, over an extended period of time.

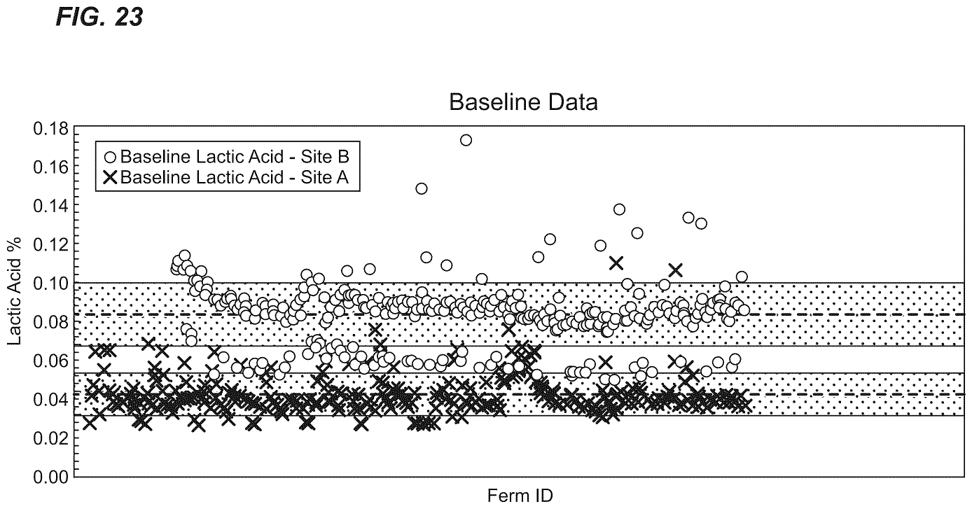

Baseline fermentation was determined over a period of time, in which the normal fermentation operations of Site A and Site B were performed. Data was collected for each operation over the course of the normal fermentation time, for example, regarding ethanol concentration, lactic acid concentration, and glucose concentration. The baseline fermentations at Site A and Site B included antibiotics. FIG. 23 shows the baseline lactic acid concentration at completion for Site A and Site B. The dotted lines extending across the plot represent the average baseline lactic acid concentrations for Site A and Site B and the solid lines above and below the dotted lines are #1 standard deviation of the average baseline lactic acid concentrations. As shown in FIG. 23, the baseline lactic acid concentration at completion was generally higher at Site B than at Site A. This suggests that Site B had higher amounts of microbial overgrowth that contributed to the higher baseline concentration of lactic acid.

A trial operation with the addition of nisin was then performed at each site. The trial at Site A included 6 fermentation tanks, each of which underwent 3 rounds of fermentation reactions, and thus 18 consecutive fermentation reactions with the added nisin were performed. As used herein, consecutive fermentation reactions means that the fermentation reactions, each with added nisin, took place one right after another (i.e. fermentation reaction 1, then fermentation reaction 2, then fermentation reaction 3, etc.). About 2.2 lbs of a nisin composition was added to each fermentation reaction: 1.1 lbs were added to the yeast propagation tank and 1.1 lbs were added to a dosing vessel which fed directly into the fermentation tank. In total, 39.6 lbs of the nisin composition were added in the 18 consecutive fermentation reactions during the trial at Site A. The nisin composition comprised about 75% nisin, which corresponds to about 30,000 IU/mg. Each fermentation tank had a volume of about 730,000 gal and was estimated to be filled to about 95% capacity for fermentation (about 693,500 gal).

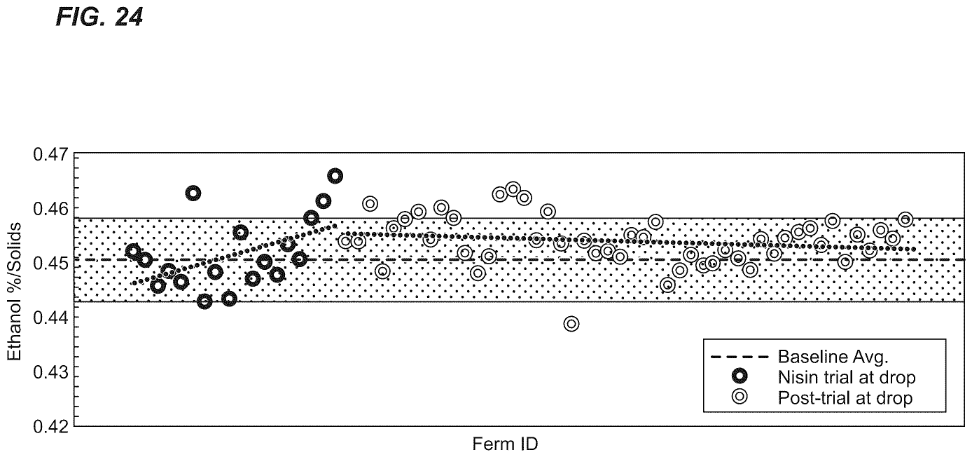

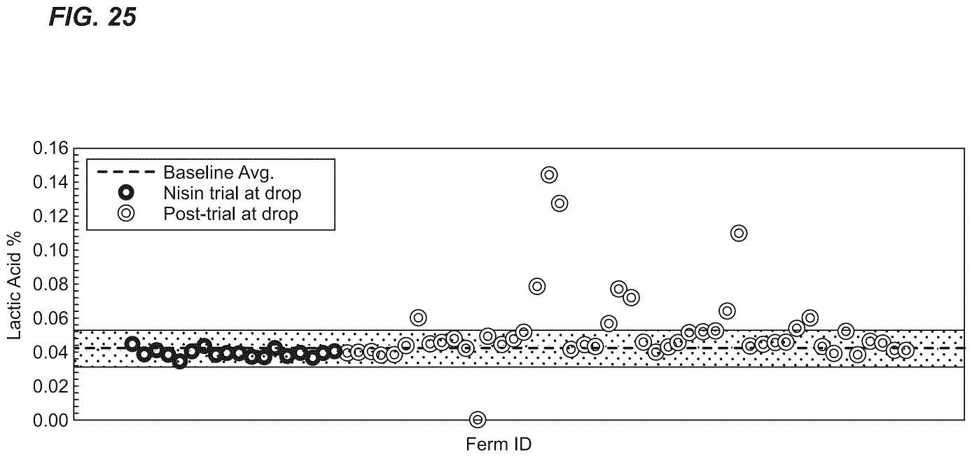

At Site A, each fermentation reaction was conducted for 66.2±6.6 hours, hereinafter referred to 66 hours for the sake of brevity, for both the baseline operation and the trial operation with nisin added. Variations in the length of each fermentation operation were due to typical factors associated with the dry grind ethanol facility and not due to the addition of nisin. As used herein, “drop” or “completion” refers to the time at which the plant empties the fermentation tank, and thus, no longer formally allows the fermentation reaction to continue. Data was measured over the course of 66 hours in the baseline period and trial operation at the following timepoints: 10-HR, 22-HR, 32-HR, 42-HR, 52-HR, 62-HR, and completion (also referred to as drop). For example, ethanol concentration, sugar concentration, lactic acid concentration, and acetic acid concentration were measured using high-performance liquid chromatography (HPLC). The results are shown in FIGS. 24-25.

FIG. 24 shows, at completion (66.2±6.6 hours), the ethanol per solids concentration for both the baseline (±1 standard deviation), post-trial baseline, and the trial operation with the added nisin, at Site A. The dotted line extending across the plot represents the average baseline final ethanol per solids concentration for Site A, and the solid lines above and below the dotted line are ±1 standard deviation of the average baseline final ethanol per solids concentration.

At completion, the fermentation trial with the added nisin generally had a final ethanol per solids concentration within the baseline (±1 standard deviation). However, as the trial continued, it appeared that the final ethanol per solids concentration also increased during the trial, as shown by the positive trend line for the data points measured during the nisin trial at drop. This suggests that there may be a backset effect. Without wishing to be bound by theory, it is believed that the nisin may be recycled into the system after the initial addition points, remaining in the recycled water supply, having a positive, long-term effect on the ethanol per solids concentration.

Also as shown in FIG. 24, this final ethanol per solids concentration was also maintained for additional fermentation reactions that occurred after the trial was completed, i.e., the nisin was no longer being added to each fermentation system, and instead the baseline treatment (antibiotics) was added. However, after nisin was no longer being added to the system, the final ethanol per solids concentration also decreased over time, as shown by the negative trend line in FIG. 24. This suggests that the addition of the nisin composition has a positive long-term effect, relative to baseline treatment, but that the positive effect decreases as additional fermentation reactions occur, and the backset effect appears to wear off when no additional nisin is added to the system.

FIG. 25 shows, at completion (66.2±6.6 hours), the lactic acid concentration for both the baseline (±1 standard deviation), the post-trial baseline, and the trial operation with the added nisin, at Site A. The dotted line extending across the plot represents the average baseline lactic acid concentration for Site A, and the solid lines above and below the dotted line are ±1 standard deviation of the average baseline lactic acid concentration. At completion, the fermentation trial with the added nisin generally had a lactic acid concentration within the baseline (±1 standard deviation), but slightly below the baseline average. After the nisin trial was completed and the baseline treatment (with antibiotics) was resumed, the lactic acid concentration remained near the baseline average for several fermentation reactions following nisin treatment. However, after several rounds of baseline treatment following the nisin treatment, the concentrations of lactic acid began to rise slightly above the baseline average. This suggests that adding nisin can provide microbial population control, through a backset effect, however, the backset effect appears to diminish after several fermentation reactions with the baseline treatment and no nisin treatment. While not wishing to be bound by theory, it is believed that the nisin composition may help decrease lactic acid concentrations, ultimately minimizing the competitive environment for the yeast. This may explain why the ethanol per solids concentration increases over the course of the trial. The spikes in the post-trial data are likely not a function of the nisin treatment or the baseline treatment, but rather an impact from external factors.

The trial at Site B included 15 fermentation tanks, each of which underwent 1 round of fermentation reactions, and thus 15 consecutive fermentation reactions with the added nisin were performed. About 2.2 lbs (1 kg) of a nisin composition was added to each fermentation reaction: 1.1 lbs were added to the yeast propagation tank and 1.1 lbs were added to a dosing vessel which fed directly into the fermentation tank. In total, 33 lbs of the nisin composition were added in the 15 consecutive fermentation reactions during the trial at Site B. The nisin composition comprised about 75% nisin, which corresponds to about 30,000 IU/mg. Each fermentation tank had a volume of about 300,000 gal and was estimated to be filled to about 95% capacity for fermentation (about 285,000 gal).

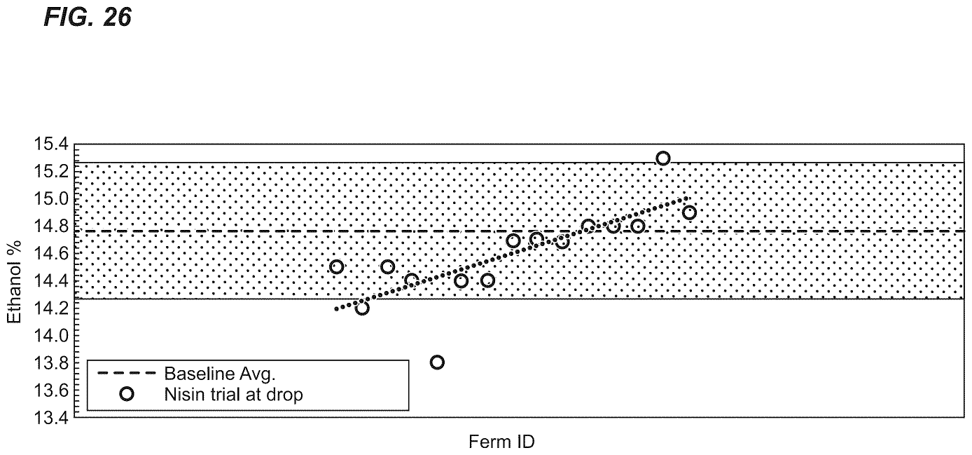

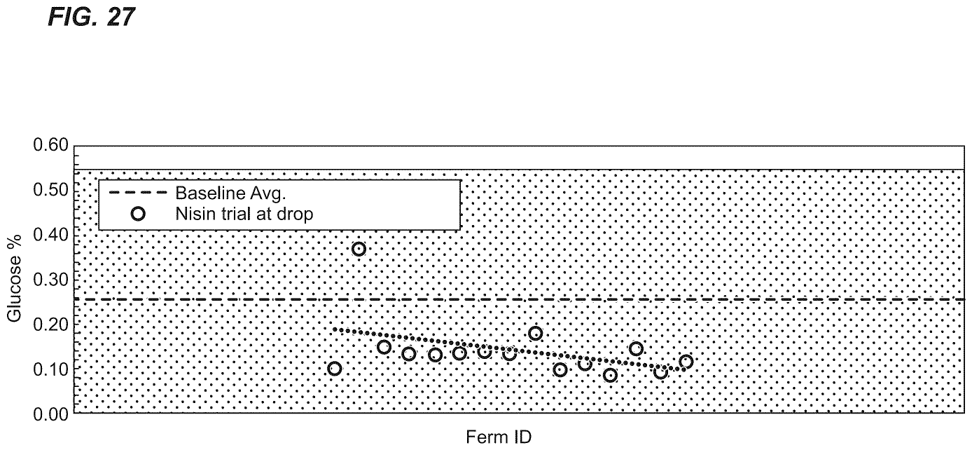

At Site B, each fermentation reaction was conducted for 62.7±3.9 hours, hereinafter referred to 63 hours for the sake of brevity, for both the baseline operation and the trial operation with nisin added. Variations in the length of each fermentation operation were due to typical factors associated with the dry grind ethanol facility and not due to the addition of nisin. Data was measured over the course of 63 hours in the baseline period and trial operation at the following timepoints: 6-HR, 12-HR, 18-HR, 24-HR, 30-HR, 36-HR, 48-HR, and completion (also referred to as drop). For example, ethanol concentration, and glucose concentration were measured using HPLC. The results are shown in FIGS. 26-27.

FIG. 26 shows, at completion (62.7±3.9 hours), the ethanol concentration for both the baseline (±1 standard deviation) and the trial operation with the added nisin, at Site B. The dotted line extending across the plot represents the average baseline final ethanol concentration for Site B and the solid lines above and below the dotted line are ±1 standard deviation of the average baseline final ethanol concentration. At completion, the fermentation trial with the added nisin generally had a final ethanol concentration within the baseline (±1 standard deviation). The final ethanol concentration generally increased in the trial fermentations, throughout the trial, as shown by the positive trend line, which suggests that the nisin composition can have positive long-term effects on the final ethanol concentration.

FIG. 27 shows, at completion (62.7±3.9 hours), the glucose concentration for both the baseline (±1 standard deviation) and the trial operation with the added nisin, at Site B. The dotted line extending across the plot represents the average baseline final glucose concentration for Site B and the solid lines above and below the dotted line are ±1 standard deviation of the average baseline final glucose concentration. At completion, the fermentation trial with the added nisin generally had a final glucose concentration within the baseline (±1 standard deviation), but below the baseline average. This suggests that more glucose was being utilized by the yeast in the fermentation reaction with the addition of nisin.

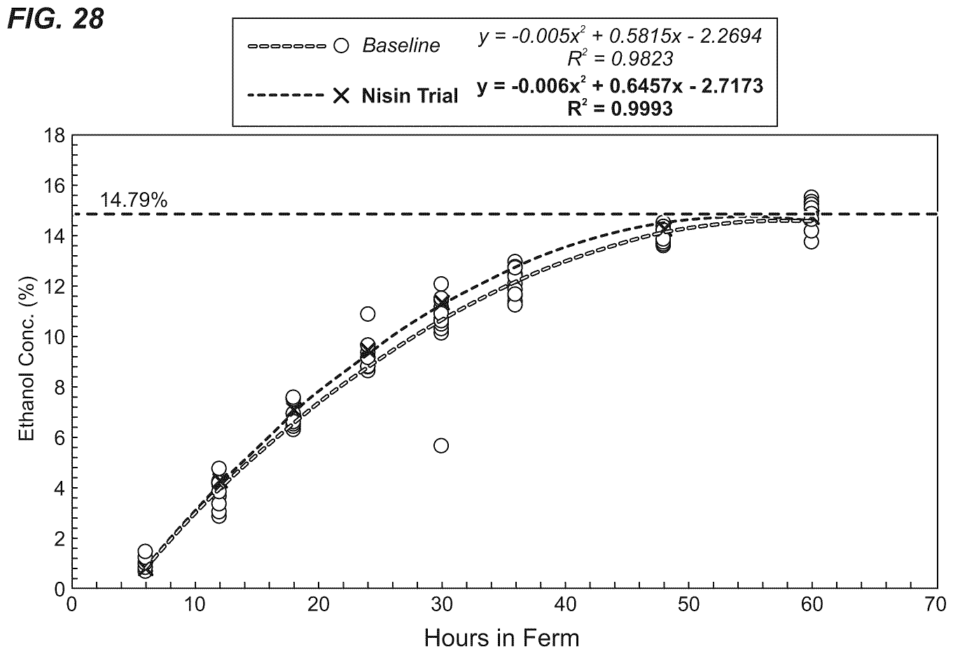

FIG. 28 shows a plot comparing the ethanol concentration in the baseline fermentation and the nisin trial over the course of fermentation at Site B. The dotted line extending across the plot represents the average baseline final ethanol concentration of 14.79% for Site B, in this fermentation vessel. After about 10 hours, the concentration of ethanol in the nisin trial begins to exceed the concentration of ethanol in the baseline fermentation, fermentation, thereby indicating the reaction comes to completion earlier. This suggests that, in some fermentation reactions, the addition of nisin can produce ethanol at a faster rate.

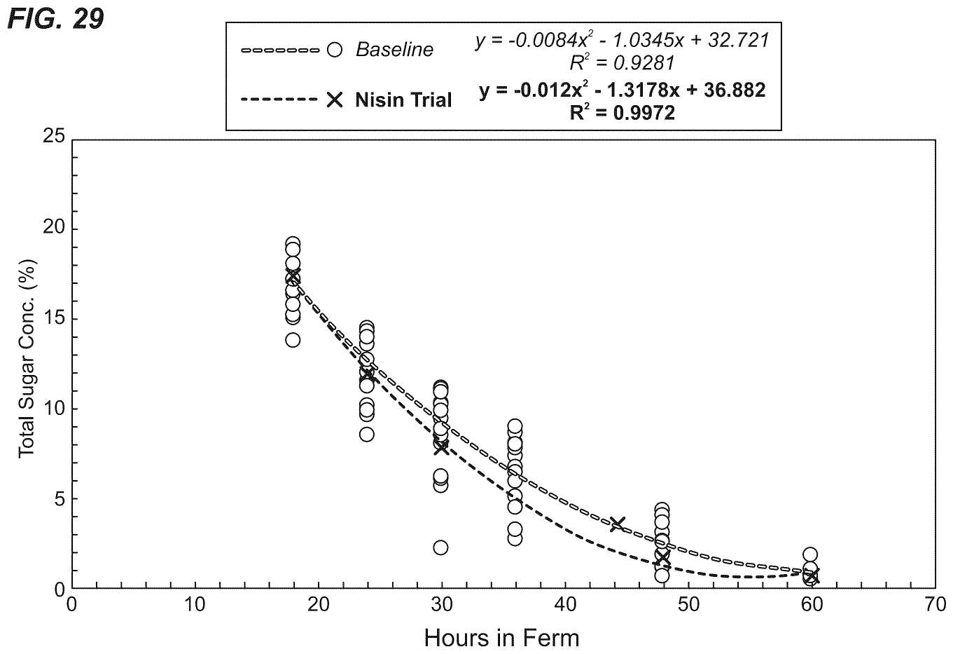

FIG. 29 shows a plot comparing the total sugar concentration in the baseline fermentation and the nisin trial over the course of the fermentation at Site B. After about 20 hours, the concentration of total sugar in the nisin trial began to decrease faster than the concentration of the total sugar in the baseline period. After about 60 hours, the concentration of total sugar in both the nisin trial and the baseline was similar. This also suggests that, in some fermentation reactions, the addition of nisin can produce ethanol at a faster rate, as the sugars are consumed more rapidly by the yeast in the fermentation reaction. Total sugars at a beginning of a fermentation reaction can have a high degree of variability therefore FIG. 29 includes data starting at 18-HR.

### Example 9: Evaluation of Wide Range of Nisin Concentrations

The present disclosure contemplates a wide range of nisin concentrations. For example, the present disclosure describes concentration ranges of 0.0001 ppm to about 75,000 ppm relative to the volume of liquid in the fermentation vessel and activities of about 1,000 IU/mg to about 41,000 IU/mg. The previous Examples 1-8 describe testing of various concentration ranges with varying activities. This Example 9 evaluates the efficacy of nisin at very high and very low concentrations with an activity of about 30,000 IU/mg.

Benchtop microbiology efficacy testing was performed for nisin using modified AOAC Method 960.09. Testing of nisin was performed against the organism of interest, Lactobacillus malefermentans. 1 mL of test organism targeting 7.0-8.0 Log CFU/mL was added to 99 mL of Nisin test solution prepared in different mediums which were adjusted to approximately pH 4.5. A nisin concentration (ppm) ladder was performed using sterile 17 grain tap water as the test medium.

Following test organism inoculation, test samples were allowed to expose at 30±1° C. and 150 RPM for 5 minutes, 4 hours and 24 hours. Following each exposure time, a 1 mL aliquot was added to 99 mL of Dey-Engley Neutralizing Broth and mixed well. Neutralized samples were serially diluted in buffer, spread plated onto MRS agar, and incubated to recover any surviving organisms. Test sample recovery was compared to untreated control samples to determine log reductions.

The nisin used in this Example 9 had an activity of about 30,000 IU/mg product and was a nisin salt blend comprising 75% nisin. This can be converted to ppm using Equation 3.

Total nisin salt blend ppm×0.75=ppm of nisin alone Eqn 3:

The results of this Example 9 are shown in FIG. 34. FIG. 34 demonstrates that nisin concentrations from 150 ppm to 75,000 ppm at about 30,000 IU/mg demonstrate a log reduction of greater than 5 at all timepoints (5 minutes, 4 hours, and 24 hours). FIG. 34 also demonstrates that very low nisin concentrations of 0.0001875 and 0.00001875 ppm demonstrate some log reduction after at least 4 hours but a log reduction of greater than 4 after 24 hours. This demonstrates that even very low concentrations of nisin at an activity about 30,000 IU/mg can demonstrate reduction and/or control of microbial populations without the use of other antimicrobials or other components to reduce and/or control microbial populations.

FIG. 4 (Example 1) of the present disclosure analyzed nisin efficacy against the same target organism also at pH 4.5 and 30° C. but with DI water instead of 17 grain tap water and at differing concentrations. Variations between expected log reduction efficacy between Examples 1 and 10 can be attributed to the use of different water sources for the testing (i.e., DI water vs. 17 grain tap water), which impacts the microbial populations to be tested and their log reduction. However, both Examples 1 and 10 demonstrate efficacy of nisin in reducing and/or controlling populations of Lactobacillus malefermentans at numerous concentrations (0.00001875-75,000 μm) and activity levels (about 1,000 IU/mg-about 30,000 IU/mg).

Having described the preferred aspects and implementations of the present disclosure, modifications and equivalents of the disclosed concepts may readily occur to one skilled in the art. However, it is intended that such modifications and equivalents be included within the scope of the claims which are appended hereto.

Also described herein are aspects:

Aspect 1: A method of reducing microbial population in a carbohydrate ethanol production system, the method comprising: adding a composition to at least one vessel in the ethanol production system, the composition comprising about 0.0001 ppm to about 3 ppm nisin relative to a volume of liquid in the at least one vessel, and the nisin having an activity of about 1,000 IU/mg to about 30,000 IU/mg, wherein the at least one vessel is selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

Aspect 2: The method of Aspect 1, wherein the composition further comprises at least one of a surfactant, one or more antimicrobial agent, a chelating agent, and a solvent.

Aspect 3: The method of Aspect 2, wherein the surfactant is selected from the group consisting of sodium xylene sulfonate, polysorbate 20, polysorbate 40, polysorbate 60, polysorbate 80, amine oxides, and combinations thereof.

Aspect 4: The method of Aspect 2, wherein the one or more antimicrobial agent is selected from the group consisting of peptide AA230, polylysine, TAED, phosphonate, thymol, sodium bisulfite, organic acids, and combinations thereof.

Aspect 5: The method of Aspect 2, wherein the chelating agent is selected from the group consisting of EDTA, DTPA, HEDTA, HEDP, MGDA, and combinations thereof.

Aspect 6: The method of any one of Aspects 1-5, wherein the composition further comprises a carrier.

Aspect 7: The method of Aspect 6, wherein the carrier is selected from the group consisting of sodium chloride, sugar, starch, sodium bicarbonate, cellulose, urea, maltose, yeast extract, sodium sulfate, calcium sulfate and combinations thereof.

Aspect 8: The method of any one of Aspects 1-7, wherein the composition is a dry powder.

Aspect 9: The method of any one of Aspects 1-7, wherein the composition is a liquid.

Aspect 10: The method of Aspect 9, wherein the composition comprises an alcohol-based or water carrier.

Aspect 11: The method of any one of Aspects 1-10, wherein the composition is free of sodium lactate and sodium acetate.

Aspect 12: The method of any one of Aspects 1-11, wherein the composition is free of antimicrobials other than nisin.

Aspect 13: The method of any one of Aspects 1-12, wherein the composition is free of biocides selected from the group consisting of 2,2-dibromo-3-nitrilopropionamide, methylene bisthiocyanate, 2-(thiocyanomethyl-thio)benzothiazole, cyanodithiocarbimate salt, N-methyldithiocarbamate salt, polyoxyethylene(dimethyliminio)-ethylene (dimethyliminio)ethylene dichloride, tetrakis(hydroxymethyl)phosphoniumsulfate, 1,1,1-tris(hydroxymethyl)nitromethane, glutaraldehyde, 1,5-pentanedial, alkylbenzyl ammonium chloride, 2-bromo-2-nitro-propane-1,3-diol, didecyl dimethyl ammonium chloride, dimethyldithiocarbamate salt, dodecylguanidine hydrochloride, 1,2-benzisothiazoline-3-one, 5-chloro-2-methyl-4-isothiazolin-3-one, 2-methyl-4-isothiazoline-3-one, n-octyl isothiazolinone, dichloro-n-octylisothiazolinone, bromonitrostyrene, or tetrahydro-3,5-dimethyl-2H-1,3,5-thiadiazine-2-thione, and combinations thereof.

Aspect 14: The method of any one of Aspects 1-13, wherein the pH of the composition is about 2 to about 9.

Aspect 15: The method of any one of Aspects 1-14, wherein the at least one vessel is the mash tank, and wherein when the composition is applied to the mash tank, the pH of mash in the mash tank with the composition added is about 2 to about 8.

Aspect 16: The method of any one of Aspects 1-15, wherein the composition is added to the ethanol production system multiple times.

Aspect 17: The method of any one of Aspects 1-15, wherein the composition is added to the ethanol production system once.

Aspect 18: The method of any one of Aspects 1-17, wherein the composition is added to at least two of the mash cooler, the mash tank, the fermentation tank, the drain, the yeast propagation tank, the liquefaction tank, the slurry tank, the strainer, the jet cooker, the hammer mill, the beerwell, and the backset.

Aspect 19: The method of any one of Aspects 1-18, wherein the nisin is a nisin salt blend.

Aspect 20: The method of Aspect 19, wherein the nisin salt blend comprises nisin and a salt comprising sodium, potassium, calcium, magnesium, ammonium, or combinations thereof.

Aspect 21: The method of Aspect 20, wherein the salt is sodium chloride, sodium bicarbonate, sodium carbonate, potassium chloride, calcium chloride, calcium carbonate, magnesium sulfate, or combinations thereof.

Aspect 22: The method of any one of Aspects 19-21, wherein the nisin salt blend comprises about 2 wt. % to about 99.99 wt. % of nisin relative to the weight of the nisin salt blend.

Aspect 23: A method for controlling microbial populations in a carbohydrate ethanol production system, the method comprising: adding nisin to at least one vessel, the at least one vessel comprising a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, wherein the nisin is the only antimicrobial agent added to the ethanol production system during a fermentation process, wherein the nisin is present from about 0.0001 ppm to about 3 ppm relative to a volume of liquid in the at least one vessel, and wherein the nisin has an activity of about 1,000 IU/mg to about 30,000 IU/mg.

Aspect 24: A method for controlling microbial populations in a carbohydrate ethanol production system, the method comprising: adding a composition comprising nisin and at least one surfactant to at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, wherein the nisin is present from about 0.0001 ppm to about 3 ppm relative to a volume of liquid in the at least one vessel, and wherein the nisin has an activity of about 1,000 IU/mg to about 30,000 IU/mg.

Aspect 25: A method of performing CIP cleaning or sanitizing of an ethanol production system, the method comprising: applying a composition to the ethanol production system, the composition comprising nisin; and circulating the composition in the ethanol production system for a period of time.

Aspect 26: The method of Aspect 25, wherein the composition is circulated in the ethanol production system for about 1 minute to about 3 hours.

Aspect 27: The method of Aspect 25 or Aspect 26, wherein after circulating the composition in the ethanol production system as part of CIP cleaning or sanitizing, the composition remains in the ethanol production system during an ethanol production operation.

Aspect 28: The method of any one of Aspects 25-27, wherein the composition further comprises one or more of peracetic acid, sodium hypochlorite, sodium hydroxide, potassium hydroxide, detergents, phosphoric acid, enzymes, hydrogen peroxide, quaternary ammonium compounds, alcohols, chelating agents, or combinations thereof.

Aspect 29: A system for monitoring microbial population in an ethanol production system, the system comprising: taking a sample from the ethanol production system; measuring the microbial population in the sample; and comparing the measured microbial population to a threshold value.

Aspect 30: The system of Aspect 29, further comprising the step of applying a composition comprising nisin to the ethanol production system prior to the step of taking a sample.

Aspect 31: The system of Aspect 29 or Aspect 30, wherein the composition comprises nisin in an amount of about 0.0001 ppm to about 3 ppm relative to a volume of liquid in a vessel that the sample is taken from, and wherein the nisin has an activity of about 1,000 IU/mg to about 30,000 IU/mg.

Aspect 32: The system of Aspect 30 or Aspect 31, wherein if the measured microbial population is higher than the threshold value, an alert is sent.

Aspect 33: The system of Aspect 32, wherein the alert is sent to a mobile device or a connected device.

Aspect 34: The system of Aspect 32 or Aspect 33, wherein the alert signals to an operator of the ethanol production system to apply a composition comprising nisin to the ethanol production system.

Aspect 35: The system of Aspect 32 or Aspect 33, wherein the alert triggers automated equipment to automatically apply a composition comprising nisin to the ethanol production system.

Aspect 36: The system of any one of Aspects 30-35, wherein the threshold value represents an upper limit of an acceptable level of microbial population.

Aspect 37: The system of any one of Aspects 30-36, wherein the sample is taken from a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

Aspect 38: A method of increasing a rate of ethanol production in a carbohydrate ethanol production system, the method comprising: adding a composition to the ethanol production system, the composition comprising about 0.0001 ppm to about 3 ppm nisin relative to a volume of liquid in at least one vessel, and the nisin having an activity of about 1,000 IU/mg to about 30,000 IU/mg, wherein the composition is added to at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and wherein the rate of ethanol production in the ethanol production system is increased relative to a system under the same or similar conditions where the composition has not been added.

Aspect 39: The method of Aspect 38, wherein the rate of ethanol production in the ethanol production system is increased by about 0.1% to about 33% relative to a system under the same or similar conditions where the composition has not been added.

Aspect 40: A method of decreasing an ethanol production time in an ethanol production system, the method comprising: adding a composition to the ethanol production system, the composition comprising about 0.0001 ppm to about 3 ppm nisin relative to a volume of liquid in at least one vessel, and the nisin having an activity of about 1,000 IU/mg to about 30,000 IU/mg, wherein the composition is added to the at least one vessel, the at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and wherein the ethanol production time in the ethanol production system is decreased relative to a system under the same or similar conditions where the composition has not been added.

Aspect 41: The method of Aspect 40, wherein the ethanol production time in the ethanol production system is decreased by about 0.1% to about 33% relative to a system under the same or similar conditions where the composition has not been added.

Aspect 42: The method of Aspect 40, wherein the ethanol production time in the ethanol production system is decreased by about 10 minutes to about 30 hours relative to a system under the same or similar conditions where the composition has not been added.

What is claimed is:

1. A method of reducing microbial population in a carbohydrate ethanol production system, the method comprising:
adding a composition to at least one vessel in the ethanol production system, the composition comprising about 0.0001 ppm to about 3 ppm nisin relative to a volume of liquid in the at least one vessel, and the nisin having an activity of about 1,000 IU/mg to about 30,000 IU/mg,
wherein the at least one vessel is selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof.

2. The method of claim 1, wherein the composition further comprises at least one of a surfactant, one or more antimicrobial agent, a chelating agent, and a solvent.

3. The method of claim 2, wherein the surfactant is selected from the group consisting of sodium xylene sulfonate, polysorbate 20, polysorbate 40, polysorbate 60, polysorbate 80, amine oxides, and combinations thereof.

4. The method of claim 2, wherein the one or more antimicrobial agent is selected from the group consisting of peptide AA230, polylysine, TAED, phosphonate, thymol, sodium bisulfite, organic acids, and combinations thereof.

5. The method of claim 2, wherein the chelating agent is selected from the group consisting of EDTA, DTPA, HEDTA, HEDP, MGDA, and combinations thereof.

6. The method of claim 1, wherein the composition further comprises a carrier.

7. The method of claim 6, wherein the carrier is selected from the group consisting of sodium chloride, sugar, starch, sodium bicarbonate, cellulose, urea, maltose, yeast extract, sodium sulfate, calcium sulfate and combinations thereof.

8. The method of claim 1, wherein the composition is a dry powder.

9. The method of claim 1, wherein the composition is free of antimicrobials other than nisin.

10. The method of claim 1, wherein the pH of the composition is about 2 to about 9.

11. The method of claim 1, wherein the composition is added to at least two of the mash cooler, the mash tank, the fermentation tank, the drain, the yeast propagation tank, the liquefaction tank, the slurry tank, the strainer, the jet cooker, the hammer mill, the beerwell, and the backset.

12. The method of claim 1, wherein the nisin is a nisin salt blend.

13. The method of claim 12, wherein the nisin salt blend comprises nisin and a salt comprising sodium, potassium, calcium, magnesium, ammonium, or combinations thereof.

14. The method of claim 13, wherein the salt is sodium chloride, sodium bicarbonate, sodium carbonate, potassium chloride, calcium chloride, calcium carbonate, magnesium sulfate, or combinations thereof.

15. A method for controlling microbial populations in a carbohydrate ethanol production system, the method comprising:
adding nisin to at least one vessel, the at least one vessel comprising a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof,
wherein the nisin is the only antimicrobial agent added to the ethanol production system during a fermentation process,
wherein the nisin is present from about 0.0001 ppm to about 3 ppm relative to a volume of liquid in the at least one vessel, and
wherein the nisin has an activity of about 1,000 IU/mg to about 30,000 IU/mg.

16. A method for controlling microbial populations in a carbohydrate ethanol production system, the method comprising:
adding a composition comprising nisin and at least one surfactant to at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof,
wherein the nisin is present from about 0.0001 ppm to about 3 ppm relative to a volume of liquid in the at least one vessel, and
wherein the nisin has an activity of about 1,000 IU/mg to about 30,000 IU/mg.

17. A method of performing CIP cleaning or sanitizing of an ethanol production system, the method comprising:
applying a composition to the ethanol production system, the composition comprising nisin; and
circulating the composition in the ethanol production system for a period of time.

18. A system for monitoring microbial population in an ethanol production system, the system comprising:
taking a sample from the ethanol production system;
measuring the microbial population in the sample; and
comparing the measured microbial population to a threshold value.

19. A method of increasing a rate of ethanol production in a carbohydrate ethanol production system, the method comprising:
adding a composition to the ethanol production system, the composition comprising about 0.0001 ppm to about 3 ppm nisin relative to a volume of liquid in at least one vessel, and the nisin having an activity of about 1,000 IU/mg to about 30,000 IU/mg,
wherein the composition is added to at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and
wherein the rate of ethanol production in the ethanol production system is increased relative to a system under the same or similar conditions where the composition has not been added.

20. A method of decreasing an ethanol production time in an ethanol production system, the method comprising:
adding a composition to the ethanol production system, the composition comprising about 0.0001 ppm to about 3 ppm nisin relative to a volume of liquid in at least one vessel, and the nisin having an activity of about 1,000 IU/mg to about 30,000 IU/mg,
wherein the composition is added to the at least one vessel, the at least one vessel selected from the group consisting of a mash cooler, a mash tank, a fermentation tank, a fermentation cooler, a drain, a yeast propagation tank, a liquefaction tank, a slurry tank, a strainer, a jet cooker, a hammer mill, a beerwell, a backset, a heat exchanger, a urea tank, a premix tank, or combinations thereof, and
wherein the ethanol production time in the ethanol production system is decreased relative to a system under the same or similar conditions where the composition has not been added.
